(19) United States
(12) Patent Application Publication
(10) Pub. No.: US 2011/0152236 A1
(43) Pub. Date: Jun. 23, 2011

(54) AZETIDINE POLYSUBSTITUTED COMPOUNDS, PREPARATION THEREOF, AND THERAPEUTIC APPLICATION THEREOF

(75) Inventors: Patrick Bernardelli, Paris (FR); Jean-Francois Sabuco, Paris (FR); Corinne Terrier, Paris (FR)

(73) Assignee: SANOFI-AVENTIS, Paris (FR)

(21) Appl. No.: 13/058,913

(22) PCT Filed: Aug. 13, 2009
(86) PCT No.: PCT/FR2009/001003

§ 371 Date: Feb. 14, 2011

(30) Foreign Application Priority Data
Aug. 14, 2008 (FR) 08/04595

(51) Int. Cl. A61K 31/427; C07D 417/12; C07D 403/12; C07D 413/12; C07D 401/12; A61K 31/4178; A61K 31/433; A61K 31/422; A61K 31/4427; A61K 31/506; A61K 31/4155; A61K 31/4245; A61K 31/4196; A61K 31/41; A61K 31/497; A61P 25/30; A61P 25/34; A61P 25/00; A61P 25/28; A61P 3/00; A61P 3/10; A61P 3/04; A61P 3/06; A61P 25/20; A61P 25/04; A61P 1/08; A61P 1/04; A61P 1/00; A61P 1/12; A61P 9/00; A61P 5/00; A61P 7/00; A61P 1/16; A61P 37/02; A61P 29/00; A61P 25/16; A61P 25/18; A61P 11/06; A61P 11/00; A61P 27/06; A61P 15/00; A61P 31/00; A61P 31/12; A61P 35/00; A61P 19/10

(52) U.S. Cl. 514/210.2; 548/195; 548/314.7; 548/139; 548/246; 546/268.1; 544/332; 548/364.1; 548/125; 548/267.6; 544/335; 548/214; 548/243; 548/254; 548/204; 544/336

(57) ABSTRACT

The invention relates to compounds of the formula (I) where: R is a (C₁-C₆)alkyl group, a halo(C₁-C₆)alkyl group; R1 is a hydrogen atom; R2 is a heteroaromatic group or a heteroaromatic(C₁-C₄)alkyl group, said groups being optionally substituted; R3 and R4 represent independently from each other an optionally substituted phenyl group; Y is a hydrogen atom, a halogen, a cyano, a (C₁-C₆)alkyl group, a halo(C₁-C₆)alkyl group, a (C₁-C₆)alkoxy group, a halo(C₁-C₆)alkoxy group or a (C₁-C₆)alkylS(O)ₚ group; and p is 0 to 2. Said compounds can be in the form_{—} of a base or a salt for addition to an acid. The invention also relates to a method for preparing same and to the therapeutic application thereof.

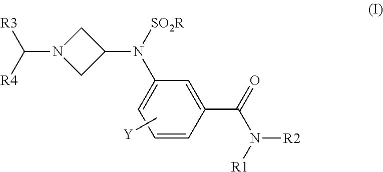

The present invention relates to azetidine derivatives, to their preparation and to their therapeutic application in the treatment or prevention of diseases involving the CB1 cannabinoid receptors.

The present invention provides compounds conforming to the formula (I)

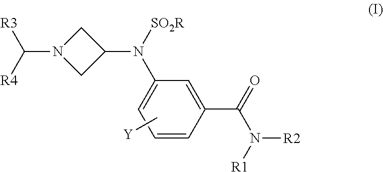

in which:
R represents a (C₁-C₆)alkyl group or a halo(C₁-C₆)alkyl group;
R1 represents a hydrogen atom;
R2 represents a

heteroaromatic group or a heteroaromatic (C₁-C₄)alkyl group, these groups being optionally substituted by one or more atoms or groups selected from a halogen, a hydroxyl, a cyano, oxo, NH₂, C(O)NH₂, a (C₁-C₆)alkyl group, a halo(C₁-C₆)alkyl group, a (C₁-C₆)alkoxy group, a halo(C₁-C₆)alkoxy group or a COO(C₁-C₆)alkyl group;
R3 and R4 represent independently of one another a phenyl group which is optionally substituted by one or more atoms or groups selected from a halogen, a cyano, a (C₁-C₆)alkyl group, a halo(C₁-C₆)alkyl group, a (C₁-C₆)alkoxy group or a halo(C1-C6)alkoxy group;
Y represents a hydrogen atom, a halogen, a cyano, a (C₁-C₆)alkyl group, a halo(C₁-C₆)alkyl group, a (C₁-C₆)alkoxy group, a halo(C₁-C6)alkoxy group or a (C₁-C₆)alkylS(O)ₚ group;
p is between 0 and 2;
   in the form of the base or an addition salt with an acid.

The compounds of formula (I) may contain one or more asymmetric carbon atoms. They may therefore exist in the form of enantiomers or diastereoisomers. These enantiomers and diastereoisomers and also mixtures thereof including the racemic mixtures, form part of the invention.

Among the compounds of formula (I) that are subjects of the invention, a first group of compounds is composed of the compounds, in a mixture of diastereoisomers and enantiomers, for which:
R represents a methyl,
R3 and R4 each represent a phenyl group which is optionally substituted by a chlorine atom in para position,
Y represents a hydrogen atom or a halogen,
R1 represents a hydrogen atom,
R2 represents a

heteroaromatic group or a heteroaromatic (C₁-C₄)alkyl group, the heteroaromatic group representing a thiazole, imidazole, thiadiazole, pyridine, isoxazole, pyrimidine, pyrazole, oxadiazole, triazole or isothiazole which is optionally substituted by one or more of (C₁-C₆)alkyl, halogen, hydroxyl, amino, C(O)NH₂, halo(C₁-C₆)alkyl;

in the form of the base or an addition salt with an acid.

The combinations of the groups mentioned above are also groups of compounds which are subjects of the invention.

In the context of the present invention

a halogen is a fluorine, a chlorine, a bromine or an iodine;

a (Cᵤ-Cₜ) represents group having from u to t carbon atoms;

a (C₁-C₆)alkyl group is an aliphatic group containing 1 to 6 carbon atoms which is saturated, cyclic, branched or linear and may optionally be substituted by one or more linear, branched or cyclic (C₁-C₆)alkyl groups. Examples include the groups methyl, ethyl, propyl, isopropyl, butyl, isobutyl, tert-butyl, pentyl, hexyl, cyclopropyl, cyclobutyl, cyclopentyl, cyclohexyl, cycloheptyl, cyclopropylmethyl, etc;

a halo(C₁-C₆)alkyl group is a (C₁-C₆)alkyl group in which one or more hydrogen atoms have been substituted by a halogen atom. Examples include the groups CF₃, CH₂CF₃, CHF₂ and CCl₃;

a hydroxy(C₁-C₆)alkyl group is a (C₁-C₆)alkyl group in which one or more hydrogen atoms have been substituted by one or more hydroxyls;

a (C₁-C₆)alkoxy group is a (C₁-C₆)alkyl-O— group in which the (C₁-C₆)alkyl group is as defined above;

a halo(C₁-C₆)alkoxy group is a halo(C₁-C₆)alkyl-O— group in which the halo(C₁-C₆)alkyl group is as defined above;

a heteroaromatic group is a 5- or 6-membered monocyclic aromatic group containing 1 to 4 heteroatoms selected from O, S and N. The N heteroatoms may be present in the oxide form, in other words N—O. Examples include

pyrrole, furan, thiophene, pyrazole, imidazole, triazole, tetrazole, oxazole, isoxazole, oxadiazole, thiazole, isothiazole, thiadiazole, pyridine, pyrimidine, pyrazine, pyridazine and triazine;

a heteroaromatic (C₁-C₄)alkyl group is an alkyl group substituted by a heteroaromatic as defined above.

The compounds of formula (I) may exist in the form of bases or salts. Addition salts of this kind form part of the invention.

These salts may be prepared with pharmaceutically acceptable acids, although the salts of other acids useful, for example, for purifying or isolating compounds of formula (I) likewise form part of the invention.

The compounds of formula (I) may also exist in the form of hydrates or solvates, namely in the form of associations or combinations with one or more molecules of water or with a solvent. Such hydrates and solvates likewise form part of the invention.

The compounds of formula (I) may also exist in the form of tautomers and likewise form part of the invention.

The compounds of formula (I) that are subjects of the invention include more particularly the following compounds; the nomenclature used corresponds to the IUPAC nomenclature.
3-[{1-[bis(4-chlorophenyl)methyl]azetidin-3-yl}(methylsulphonyl)amino]-N-(1,3-thiazol-2-benzamide
3-[{1-[bis(4-chlorophenyl)methyl]azetidin-3-yl}(methylsulphonyl)amino]-N-[2-(1H-imidazol-1-yl)ethyl]benzamide
3-[{1-[bis(4-chlorophenyl)methyl]azetidin-3-yl}(methylsulphonyl)amino]-N-(1,3,4-thiadiazol-2-yl)benzamide
3-[{1-[bis(4-chlorophenyl)methyl]azetidin-3-yl}(methylsulphonyl)amino]-N-(4-hydroxy-1-methyl-1H-imidazol-2-yl)benzamide
3-[{1-[bis(4-chlorophenyl)methyl]azetidin-3-yl}(methylsulphonyl)amino]-N-(isoxazol-3-yl)-benzamide
3-[{1-[bis(4-chlorophenyl)methyl]azetidin-3-yl}(methylsulphonyl)amino]-N-(5-cyclopropyl-1,3,4-thiadiazol-2-yl)benzamide
3-[{1-[bis(4-chlorophenyl)methyl]azetidin-3-yl}(methylsulphonyl)amino]-N-(pyridin-2-yl)-benzamide
3-[{1-[bis(4-chlorophenyl)methyl]azetidin-3-yl}(methylsulphonyl)amino]-N-(pyrimidin-2-yl)-benzamide
3-[{1-[bis(4-chlorophenyl)methyl]azetidin-3-yl}(methylsulphonyl)amino]-N-(1H-pyrazol-3-yl)-benzamide
N-(4-amino-1,2,5-oxadiazol-3-yl)-3-[{1-[bis(4-chlorophenyl)methyl]azetidin-3-yl}(methylsulphonyl)amino]benzamide
3-[{1-[bis(4-chlorophenyl)methyl]azetidin-3-yl}(methylsulphonyl)amino]-N-(4-hydroxypyridin-2-yl)benzamide
3-[({3-[{1-[bis(4-chlorophenyl)methyl]azetidin-3-yl}(methylsulphonyl)amino]phenyl}carbonyl)-amino]-1H-pyrazole-4-carboxamide
3-[{1-[bis(4-chlorophenyl)methyl]azetidin-3-yl}(methylsulphonyl)amino]-N-(1H-1,2,4-triazol-3-ylmethyl)benzamide
3-[{1-[bis(4-chlorophenyl)methyl]azetidin-3-yl}(methylsulphonyl)amino]-N-(1H-pyrazol-3-ylmethyl)benzamide
3-[{1-[bis(4-chlorophenyl)methyl]azetidin-3-yl}(methylsulphonyl)amino]-N[2-(1H-pyrazol-1-yl)ethyl]benzamide
3-[{1-[bis(4-chlorophenyl)methyl]azetidin-3-yl}(methylsulphonyl)amino]-N-[2-(pyrimidin-2-yl)-ethyl]benzamide
3-[{1-[bis(4-chlorophenyl)methyl]azetidin-3-yl}(methylsulphonyl)amino]-5-fluoro-N-(isoxazol-3-yl)benzamide
3-[{1-[bis(4-chlorophenyl)methyl]azetidin-3-yl}(methylsulphonyl)amino]-5-fluoro-N-(pyridin-2-yl)benzamide
3-[{1-[bis(4-chlorophenyl)methyl]azetidin-3-yl}(methylsulphonyl)amino]-5-fluoro-N-(1,3-thiazol-2-yl)benzamide
3-[{1-[bis(4-chlorophenyl)methyl]azetidin-3-yl}(methylsulphonyl)amino]-5-fluoro-N-(pyrimidin-2-yl)benzamide
3-[{1-[bis(4-chlorophenyl)methyl]azetidin-3-yl}(methylsulphonyl)amino]-5-fluoro-N-(5-methylisoxazol-3-yl)benzamide
3-[{1-[bis(4-chlorophenyl)methyl]azetidin-3-yl}(methylsulphonyl)amino]-5-fluoro-N-[4-(trifluoromethyl)-1,3-thiazol-2-yl]benzamide
3-[{1-[bis(4-chlorophenyl)methyl]azetidin-3-yl}(methylsulphonyl)amino]-5-fluoro-N-(3-methylisothiazol-5-yl)benzamide
3-[{1-[bis(4-chlorophenyl)methyl]azetidin-3-yl}(methylsulphonyl)amino]-5-fluoro-N-(isoxazol-4-yl)benzamide
3-[{1-[bis(4-chlorophenyl)methyl]azetidin-3-yl}(methylsulphonyl)amino]-5-fluoro-N-(4-methyl-1,3-thiazol-2-yl)benzamide
3-[{1-[bis(4-chlorophenyl)methyl]azetidin-3-yl}(methylsulphonyl)amino]-5-fluoro-N-(3-methylisoxazol-5-yl)benzamide
3-[{1-[bis(4-chlorophenyl)methyl]azetidin-3-yl}(methylsulphonyl)amino]-5-fluoro-N-(4-methylpyridin-2-yl)benzamide
3-[{1-[bis(4-chlorophenyl)methyl]azetidin-3-yl}(methylsulphonyl)amino]-5-fluoro-N-(6-methylpyridin-2-yl)benzamide
3-[{1-[bis(4-chlorophenyl)methyl]azetidin-3-yl}(methylsulphonyl)amino]-5-fluoro-N-(1H-pyrazol-3-yl)benzamide
N-(6-aminopyridin-3-yl)-3-[{1-[bis(4-chlorophenyl)methyl]azetidin-3-yl}(methylsulphonyl)amino]benzamide
N-(3-amino-1H-1,2,4-triazol-5-yl)-3-[{1-[bis(4-chlorophenyl)methyl]azetidin-3-yl}(methylsulphonyl)amino]benzamide
3-[{1-[bis(4-chlorophenyl)methyl]azetidin-3-yl}(methylsulphonyl)amino]-N-[(3-hydroxyisoxazol-5-yl)methyl]benzamide
3-[{1-[bis(4-chlorophenyl)methyl]azetidin-3-yl}(methylsulphonyl)amino]-N-(2H-tetrazol-5-ylmethyl)benzamide
3-({1-[bis(4-chlorophenyl)methyl]azetidin-3-yl}methylsulphonylamino)-N-(4-cyanopyridin-2-yl)-5-fluorobenzamide
3-({1-[bis(4-chlorophenyl)methyl]azetidin-3-yl}methylsulphonylamino)-5-fluoro-N-pyridin-2-ylmethylbenzamide
3-({1-[bis(4-chlorophenyl)methyl]azetidin-3-yl}methylsulphonylamino)-5-fluoro-N-(pyridin-3-ylmethyl)benzamide
3-({1-[bis(4-chlorophenyl)methyl]azetidin-3-yl}methylsulphonylamino)-5-fluoro-N-[1-(pyridin-3-yl)ethyl]benzamide
3-({1-[bis(4-chlorophenyl)methyl]azetidin-3-yl}methylsulphonylamino)-5-fluoro-N-[2-(pyridin-3-yl)propyl]benzamide
3-({1-[bis(4-chlorophenyl)methyl]azetidin-3-yl}methylsulphonylamino)-5-fluoro-N-[1-(pyridin-2-yl)ethyl]benzamide
3-({1-[bis(4-chlorophenyl)methyl]azetidin-3-yl}methylsulphonylamino)-5-fluoro-N-[(2-methylthiazol-4-yl)methyl]benzamide
3-({1-[bis(4-chlorophenyl)methyl]azetidin-3-yl}methylsulphonylamino)-5-fluoro-N[2-([1,2,4]-triazol-1-yl)propyl]benzamide
3-({1-[bis(4-chlorophenyl)methyl]azetidin-3-yl}methylsulphonylamino)-5-fluoro-N-[1-(2-methylthiazol-4-yl)ethyl]benzamide
3-({1-[bis(4-chlorophenyl)methyl]azetidin-3-yl}methylsulphonylamino)-5-fluoro-N-[1-(1-methyl-1H-pyrazol-4-yl)ethyl]benzamide
3-({1-[bis(4-chlorophenyl)methyl]azetidin-3-yl}methylsulphonylamino)-5-fluoro-N-(pyridin-3-yl)benzamide
3-({1-[bis(4-chlorophenyl)methyl]azetidin-3-yl}methylsulphonylamino)-5-fluoro-N-[1-(2-pyrazol-1-yl)propyl]benzamide
3-({1-[bis(4-chlorophenyl)methyl]azetidin-3-yl}methylsulphonylamino)-5-fluoro-N-(pyridin-4-ylmethyl)benzamide
3-({1-[bis(4-chlorophenyl)methyl]azetidin-3-yl}methylsulphonylamino)-5-fluoro-N-[(6-oxo-1,6-dihydropyridin-3-yl)methyl]benzamide
3-({1-[bis(4-chlorophenyl)methyl]azetidin-3-yl}methylsulphonylamino)-5-fluoro-N-[(1-pyridin-4-yl)ethyl]benzamide
3-({1-[bis(4-chlorophenyl)methyl]azetidin-3-yl}methylsulphonylamino)-N-[(6-dimethylaminopyridin-3-yl)methyl]-5-fluorobenzamide
3-({1-[bis(4-chlorophenyl)methyl]azetidin-3-yl}methylsulphonylamino)-5-fluoro-N-[2-(pyrazin-2-yl)propyl]benzamide
3-({1-[bis(4-chlorophenyl)methyl]azetidin-3-yl}methylsulphonylamino)-5-fluoro-N-(pyridin-4-yl)benzamide
3-({1-[bis(4-chlorophenyl)methyl]azetidin-3-yl}methylsulphonylamino)-5-fluoro-N-[2-hydroxy-2-(pyridin-4-yl)ethyl]benzamide
   and their pharmaceutically acceptable salts.

The present invention likewise provides for the use of the compounds of the invention of formula (I) for preparing a medicament for treating or preventing diseases in which the CB1 receptor is involved.

The present invention likewise provides for the use of the compounds of the invention of formula (1) for preparing a medicament for treating or preventing psychiatric disorders, dependence on and withdrawal from a substance, tobacco withdrawal, cognitive disorders and attention disorders, and acute and chronic neurodegenerative diseases; the metabolism, appetence disorders, appetite disorders, obesity, diabetes (type I and/or II), metabolic syndrome, dyslipidaemia or sleep apnoea; pgin, neuropathic nr neuropathic pain induced by anti-cancer agents; gastrointestinal disorders, vomiting, ulcers, diarrhoea disorders, bladder and urinary disorders, disorders of endocrine origin, cardiovascular disorders, hypotension, haemorrhagic shock, septic shock, liver diseases, chronic liver cirrhosis, fibrosis, non-alcoholic steatohepatitis (NASH), steatohepatitis and hepatic steatosis, irrespective of the etiology of these conditions (alcohol, medicament, chemical product, autoimmune disease, obesity, diabetes, congenital metabolic disease); diseases of the immune system, rheumatoid arthritis, demyelination, multiple sclerosis or inflammatory diseases; Alzheimer's disease, Parkinson's disease, schizophrenia or cognitive disorders associated with schizophrenia, with diabetes, with obesity or with metabolic syndrome; asthma, chronic obstructive pulmonary diseases, Raynaud syndrome, glaucoma or fertility disorders; infectious and viral diseases such as encephalitis, cerebral strokes, Guillain-Barré syndrome, osteoporosis and sleep apnoea and for anti-cancer chemotherapy; and disorders linked with anti-psychotic treatments (weight gain, metabolic disorder).

In accordance with the invention, the compounds of general formula (I) can be prepared in accordance with the process described in scheme 1:

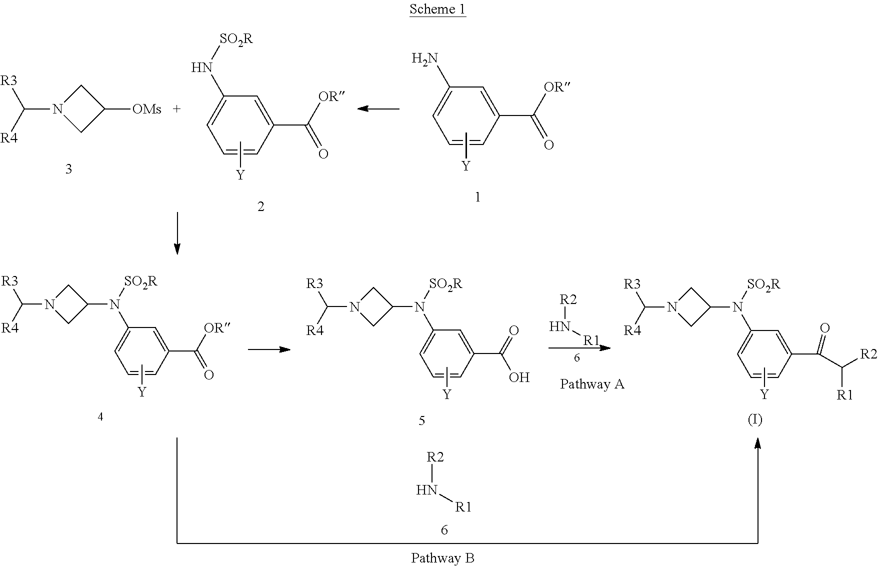

The mesylation of compound 1 to derivative 2 can be carried out according to the methods which are known to the skilled person or else are described in T. W. Greene, Protective Groups in Organic Synthesis, fourth edition. This reaction takes place in a chlorinated solvent such as dichloromethane, in the presence of a base such as pyridine and a mesylate derivative such as mesyl chloride, at a temperature between −10° C. and 40° C.

The derivatives 1 are available commercially or synthesized, by methods which are known to the skilled person, from appropriate commercial precursors; R″ represents a protective group for the OH function of the acid.

The derivative 4 is accessible by reaction of the mesylate 2 with the azetidine 3. This step takes place preferably under an inert atmosphere, within an inert solvent such as 4-methyl-2-pentanone in the presence of an inorganic base such as potassium carbonate, at the reflux of the reaction mixture.

The synthesis of the azetidine 3 is described in patent application WO01064634.

The hydrolysis of the ester 4 to acid 5 takes place in accordance with the methods which are known to the skilled person, and more particularly in a mixture of polar solvents such as tetrahydrofuran and water in the presence of a base such as lithium hydroxide hydrate, at a temperature in the region of 20° C.

The formation of the compounds of formula (I) may take place:
by pathway A, by reaction of the acid 5 with an amine derivative 6. This reaction may be carried out:

within a chlorinated solvent such as dichloromethane, in the presence of a coupling agent such as 1-(3-dimethylaminopropyl)-3-ethylcarbodiimide hydrochloride;

within a polar solvent such as tetrahydrofuran or dimethylformamide, in the presence of a base such as a trialkylamine (triethylamine or a diisopropylethylamine, for example), in the presence or absence of a coupling agent such as 1-(3-dimethylaminopropyl)-3-ethylcarbodiimide hydrochloride, in the presence of one or more additives (1-hydroxybenzotriazole, benzotriazol-1-yloxytris(dimethylamino)phosphonium hexafluoride, for example);

within a polar solvent such as tetrahydrofuran, in the presence of a base such as a trialkylamine (triethylamine, for example), in the presence of an agent allowing peptide synthesis via the formation of a mixed anhydride, such as isobutyl chloroformate;

within a polar solvent such as tetrahydrofuran or a chlorine solvent such as dichloromethane, in the presence of an aliquot of dimethylformamide, in the presence of an agent allowing the intermediate formation of an acid chloride (thionyl chloride, for example) and at a temperature between −20° C. and the boiling temperature of the solvent;
by pathway B, by reaction of the ester 4 with an amine derivative 6. This reaction may be carried out within an inert solvent such as toluene, in the presence of a trialkylaluminium derivative such as trimethylaluminium at a temperature between 0° C. and the boiling temperature of the solvent.

The derivatives 6 are available commercially or are synthesized, by methods which are known to the skilled person, from appropriate commercial precursors.

The compounds of formula (I) may be prepared by reacting an acid derivative 5 with an amine derivative 6 in an inert solvent, in the presence of a coupling agent and optionally of an additive which prevents racemization; the product is deprotected, where appropriate, and then the product is isolated and is optionally converted to an addition salt with an acid.

The compounds of formula (I) may be purified by customary known methods, for example by crystallization, chromatography or extraction.

The enantiomers of the compounds of formula (I) may be obtained by resolving the racemic mixtures, for example by chromatography on a chiral column according to W. H. Pirkle et al., Asymmetric Synthesis, vol. 1, Academic Press (1983), or by formation of salts or by synthesis from chiral precursors. The diastereoisomers may be prepared by the conventional known methods (crystallization, chromatography or from chiral precursors).

The present invention also relates to the process for preparing the intermediates.

The examples which follow describe the preparation of certain compounds in accordance with the invention. These examples are not limitative and merely illustrate the present invention. The numbers of the compounds exemplified match with those given in the table hereinafter, which illustrates the chemical structures and the physical properties of some compounds according to the invention.

### EXAMPLES

### Example 1

### 3-[{1-[Bis(4-chlorophenyl)methyl]azetidin-3-yl}(methylsulphonyl)amino]-N-(1,3-thiazol-2-yl)benzamide (Compound 1)

A solution of 300 mg of 3-[{1-[bis(4-chlorophenyl)methyl]azetidin-3-yl}(methylsulphonyl)amino]benzoic acid and 65.4 mg of 2-aminothiazole in 3 cm³ of dichloromethane, stirred for 10 minutes at a temperature in the region of 20° C., is admixed with 136.5 mg of 1-(3-dimethylaminopropyl)-3-ethylcarbodiimide hydrochloride. The reaction mixture is stirred overnight at a temperature in the region of 20° C. before being diluted with water. The aqueous phase is extracted with dichloromethane. The organic phases are combined, dried over magnesium sulphate, filtered and concentrated to dryness under reduced pressure to give a crude product which is purified by flash chromatography on a Sep Pack column of 5 g of silica (elution gradient: dichloromethane/methanol 100/0 to 96/4). Concentration of the fractions under reduced pressure gives 170 mg of 3-[{1-[bis(4-chlorophenyl)methyl]azetidin-3-yl}(methylsulphonyl)amino]-N-(1,3-thiazol-2-yl)benzamide in the form of white crystals.

m.p.: 244° C.

1H NMR spectrum (400 MHz; (δ in ppm); (DMSO-d6); referenced at 2.50 ppm): 2.74 (m, 2H); 3.00 (s, 3H); 3.37 (m, 2H); 4.39 (s, 1H); 4.74 (m, 1H); 7.27 (masked m, 1H); 7.29 (d, J=8.4 Hz, 4H); 7.35 (d, J=8.4 Hz, 4H); 7.51-7.61 (m, 3H); 8.02-8.08 (m, 2H); 12.72 (extended m, 1H)

Mass spectrum: ES m/z=587 (M+1)⁺; m/z=585 (M−H)⁻

Elemental analysis:

Calculated: C: 55.20%-H: 4.12%-N: 9.54%-S: 10.91%

Measured: C: 53.99%-H: 4.10%-N: 9.02%-S: 10.12%-H₂O: 2.65%

### Example 2

### 3-[{1-[Bis(4-chlorophenyl)methyl]azetidin-3-yl}(methylsulphonyl)amino]-N-(pyridin-2-yl)benzamide (Compound 7)

A solution of 300 mg of 3-[{1-[bis(4-chlorophenyl)methyl]azetidin-3-yl}(methylsulphonyl)amino]benzoic acid methyl ester and 3 cm³ of trirnethylaluminium in 5 cm³ of toluene is admixed with 90.6 mg of 2-aminopyridine. The reaction mixture, placed in a Radley tube, is stirred overnight at a temperature in the region of 50° C. before being diluted with water. The aqueous phase is extracted with dichloromethane in a hydrophobic filtration syringe. The crude product is chromatographed on a column of silica of 30 g (eluent: dichloromethane/methanol 90/10). Concentration of the fractions under reduced pressure gives 50 mg of 3-[{1-[bis(4-chlorophenyl)methyl]azetidin-3-yl}(methylsulphonyl)amino]-N-(pyridin-2-yl)benzamide in the form of beige crystals.

m.p.: 202° C.

1H NMR spectrum (400 MHz; (δ in ppm); (DMSO-d6); referenced at 2.50 ppm): 2.75 (m, 2H); 3.00 (s, 3H); 3.36 (m, 2H); 4.39 (s, 1H); 4.75 (m, 1H); 7.18 (m, 1H); 7.30 (d, J=8.8 Hz, 4H); 7.35 (d, J=8.8 Hz, 4H); 7.51-7.57 (m, 2H); 7.85 (m, 1H); 7.97 (s,)H); 8.00 (m, 1H); 8.18 (d, J=8.4 Hz, 1H); 8.40 (broad d, J=5.0 Hz, 1H); 10.89 (broad s, 1H);

Mass spectrum: ES m/z=581 [M+H]⁺; m/z=347 [M+H−C₁₃H₈Cl₂]⁺; m/z=579 [M−H]⁻; m/z=1159 [2M−H]⁻

### Example 3

### 3-[{1-[Bis(4-chlorophenyl)methyl]azetidin-3-yl}(methylsulphonyl)amino]-N-(1H-1,2,4-triazol-3-ylmethyl)benzamide (Compound 13)

A solution of 500 mg of 3-[{1-[bis(4-chlorophenyl)methyl]azetidin-3-yl}-(methylsulphonyl)amino]benzoic acid, 66.8 mg of 1-hydroxybenzotriazole, 0.138 cm³ of triethylamine and 265 mg of 1-(3-dimethylaminopropyl)-3-ethylcarbodiimide hydrochloride in 15 cm³ of tetrahydrofuran is admixed dropwise with 97 mg of 1H-1,2,4-triazole-3-methanamine. The reaction mixture is stirred overnight at a temperature in the region of 20° C. before being concentrated to dryness under reduced pressure. The residue is taken up in a dichloromethane/water mixture. Following phase separation, the organic phase is dried over magnesium sulphate, filtered and then concentrated to dryness under reduced pressure. The crude reaction product obtained is purified by flash chromatography on a column of 30 g of silica (Merck; elution gradient: dichloromethane/methanol 100/0 to 95/5). Concentration of the fractions under reduced pressure gives 320 mg of 3-[{1-[bis(4-chlorophenyl)methyl]azetidin-3-yl)(methylsulphonyl)amino]-N-(1H-1,2,4-triazol-3-ylmethyl)benzamide in the form of white crystals.

m.p.: 210° C.

1H NMR spectrum (400 MHz; (δ in ppm); (DMSO-d6); referenced at 2.50 ppm): 2.70 (m, 2H); 2.97 (s, 3H); 3.34 (partially masked m, 2H); 4.37 (s, 1H); 4.55 (d, J=5.9 Hz, 2H); 4.73 (m, 1H); 7.31 (d, J=8.8 Hz, 4H); 7.35 (d, J=8.8 Hz, 4H); 7.46-7.54 (m, 2H); 7.80 (broad s, 1H); 7.87 (m, 1H); 8.17 (extended m, 1H); 9.10 (broad t, J=5.6 Hz, 1H); 13.80 (very extended m, 1H)

Mass spectrum: ES m/z=585 [M+H]⁺; m/z=583 [M−H]⁻

### Example 4

### 3-[{1-[Bis(4-chlorophenyl)methyl]azetidin-3-yl}(methylsulphonyl)amino]-N-(1H-pyrazol-3-ylmethyl)benzamide (Compound 14)

A solution of 400 mg of 3-[{1-[bis(4-chlorophenyl)methyl]azetidin-3-yl}-(methylsulphonyl)amino]benzoic acid, 55 mg of 1-hydroxybenzotriazole, 0.113 cm³ of triethylamine and 213 mg of 1-(3-dimethylaminopropsyl)-3-ethylcarbodiimide hydrochloride in tetrahydrofuran is admixed dropwise with 77 mg of 1H-pyrazol-3-ylmethylamine. The reaction mixture is stirred overnight at a temperature in the region of 20° C. before being concentrated to dryness under reduced pressure. The residue is purified by flash chromatography on a column of 30 g of silica (Merck; elution gradient: dichloromethane/methanol 100/0 to 96/4). Concentration of the fractions under reduced pressure gives 390 mg of 3-[{1-[bis(4-chlorophenyl)methyl]azetidin-3-yl}(methylsulphonyl)amino]-N-(1H-pyrazol-3-ylmethyl)benzamide in the form of white crystals.

m.p.: 224° C.

1H NMR spectrum (400 MHz; (δ in ppm); (DMSO-d6); referenced at 2.50 ppm): 2.70 (m, 2H); 2.96 (s, 3H); 3.33 (m, 2H); 4.37 (s, 1H); 4.47 (extended m, 2H); 4.72 (m, 1H); 6.16 (broad s, 1H); 7.30 (d, J=8.7 Hz, 4H); 7.35 (d, J=8.7 Hz, 4H); 7.44-7.67 (m, 3H); 7.79 (broad s, 1H); 7.86 (m, 1H); 8.96 (extended m, 1H); 12.57 (extended m, 1H)

Mass spectrum: ES m/z=584 ([M+H]⁺, base peak); m/z=350 [M+H−C₁₃H₈Cl₂]⁺

### Example 5

### 3-[{1-[Bis(4-chlorophenyl)methyl]azetidin-3-yl}(methylsulphonyl)amino]-5-fluoro-N-(pyrimidin-2-yl)benzamide (Compound 20)

### 5a: 3-Fluoro-5-methanesulphonylaminobenzoic acid ethyl ester

A solution of 4 g of 5-amino-3-fluorobenzoic acid ethyl ester in 100 cm³ of dichloromethane, stirred under an argon atmosphere, is admixed with 2.65 cm³ of pyridine. The reaction mixture is cooled to a temperature in the region of 0° C. by means of an ice bath, and then a solution of 1.78 cm³ of methanesulphonyl chloride in 2 cm³ of dichloromethane is added dropwise. The orange-coloured solution obtained is allowed to return to a temperature in the region of 20° C. and is stirred at this temperature for 20 h. Following addition of 40 cm³ of distilled water and 50 cm³ of dichloromethane, followed by phase separation, the organic phase is washed in succession with 35 cm³ of distilled water and then 40 cm³ of saturated aqueous sodium chloride solution. The organic phase is dried over sodium sulphate, filtered on a glass frit and then concentrated to dryness under reduced pressure to give 5.8 g of an orange-coloured solid. The crude reaction product is purified by flash chromatography on a cartridge of 400 g of Merck silica (particle size: 15-40 μm; eluent: dichloromethane/methanol 98/2). Concentration of the fractions under reduced pressure gives 5.09 g of 3-fluoro-5-methanesulphonylaminobenzoic acid ethyl ester in the form of a white solid.

Mass spectrum: EI m/z=261 (M⁺, base peak), m/z=233 [(M−C₂H₄)⁺], m/z=216 [(M−OC₂H₅)⁺], m/z=182 [(M−SO₂CH₃)⁻], m/z=138 [(m/z=182−OC₂H₄)⁺]

### 5b: 3-[{1-[Bis(4-chlorophenyl)methyl]azetidin-3-yl}(methylsulphonyl)amino]-5-fluorobenzoic acid ethyl ester

A suspension of 3.7 g of 1-[bis(4-chlorophenyl)methyl]azetidin-3-yl methanesulphonate and 3.5 g of 3-fluoro-5-methanesulphonylaminobenzoic acid ethyl ester in 130 cm³ of 4-methyl-2-pentanone is admixed with 3.97 g of potassium carbonate. The reaction mixture is stirred at reflux for 7 hours and then allowed to return to a temperature in the region of 20° C. for 16 hours. The resulting cream suspension is admixed with 50 cm³ of distilled water and 100 cm³ of ethyl acetate. After 30 minutes of stirring, followed by phase separation, the aqueous phase is extracted with twice 100 cm³ of ethyl acetate. The combined organic phases are washed with 80 cm³ of saturated aqueous sodium chloride solution, dried over sodium sulphate, filtered on a glass frit and then concentrated to dryness under reduced pressure to give 7.2 g of an orange-coloured residue. The crude reaction product is purified by flash chromatography on a cartridge of 400 g of Merck silica (particle size: 15-40 μm; elution gradient: dichloromethane/methanol 98/2 to 95/5). Concentration of the fractions under reduced pressure gives 4.03 g of 3-[{1-[bis(4-chlorophenyl)methyl]azetidin-3-yl}(methylsulphonyl)amino]-5-fluorobenzoic acid ethyl ester in the form of a white foam.

1H NMR spectrum (400 MHz; (δ in ppm); (DMSO-d6); referenced at 2.50 ppm): 1.32 (t, J=7.2 Hz, 3H); 2.73 (t, J=7.3 Hz, 2H); 2.98 (s, 3H); 3.35 (m, 2H); 4.34 (q, J=7.2 Hz, 2H); 4.43 (s, 1H); 4.77 (m, 1H); 7.31 (d, J=8.8 Hz, 4H); 7.37 (d, J=8.8 Hz, 4H); 7.56 (dt, J=9.8; 2.4 Hz, 1H); 7.66 (broad d, J=9.1 Hz, 1H); 7.70 (broad s, 1H)

Mass spectrum: ES m/z=551 (MH⁺), m/z=235 (C₁₃H₉Cl₂⁺, base peak)

### 5c: 3-[{1-[Bis(4-chlorophenyl)methyl]azetidin-3-yl}(methylsulphonyl)amino]-5-fluorobenzoic acid

A solution of 2.5 g of 3-[{1-[bis(4-chlorophenyl)methyl]azetidin-3-yl}(methylsulphonyl)amino]-5-fluorobenzoic acid ethyl ester in a mixture composed of 34 cm³ of tetrahydrofuran and 9 cm³ of water, stirred under an argon atmosphere, is admixed with 0.222 g of lithium hydroxide in two portions. The reaction mixture is stirred at a temperature in the region of 20° C. for 24 hours. Then 100 cm³ of saturated aqueous sodium hydrogenphosphate solution are added to bring the pH to 5. The aqueous phase is extracted with four times 200 cm³ of ethyl acetate. The combined organic phases are dried over sodium sulphate, filtered on a glass frit and then concentrated to dryness under reduced pressure to give a foam which is taken up twice in 150 cm³ of ethyl ether. Concentration under reduced pressure gives 2.3 g of 3-[{1-[bis(4-chlorophenyl)methyl]azetidin-3-yl}(methylsulphonyl)amino]-5-fluorobenzoic acid in the form of a white solid.

1H NMR spectrum (400 MHz; (δ in ppm); (DMSO-d6); referenced at 2.50 ppm): 2.74 (t, J=6.9 Hz, 2H); 2.98 (s, 3H); 3.33 (masked m, 2H); 4.43 (s, 1H); 4.76 (quin, J=6.9 Hz, 1H); 7.31 (d, J=8.8 Hz, 4H); 7.36 (d, J=8.8 Hz, 4H); 7.51 (dt, J=9.4; 2.0 Hz, 1H); 7.64 (dt, J=8.9; 2.0 Hz, 1H); 7.70 (t, J=2.0 Hz, 1H); 13.25 (very extended m, 1H)

Mass spectrum: ES m/z=523 (M+H), m/z=235 (C₁₃H₉Cl₂⁺, base peak)

### 5d: 3-[{1-[Bis(4-chlorophenyl)methyl]azetidin-3-yl}(methylsulphonyl)amino]-5-fluoro-N-(pyrimidin-2-yl)benzamide (Compound 20)

A solution of 500 mg of 3-[{1-[bis(4-chlorophenyl)methyl]azetidin-3-yl}(methylsulphonyl)amino]-5-fluorobenzoic acid in 10 cm³ of dichloromethane is admixed in succession with 4 drops of dimethylformamide and then a solution of 400 μl of thionyl chloride in 1 cm³ of dichloromethane. The reaction mixture is stirred for 45 minutes at a temperature in the region of 20° C. before being concentrated to dryness under reduced pressure after addition of a few cm³ of toluene. The resulting residue is dissolved in 5 cm³ of dichloromethane. This solution is admixed in succession with a solution of 109 mg of 2-aminopyrimidine in a tetrahydrofuran 4 cm³/dichloromethane 4 cm³ mixture, then 400 μl of triethylamine. The reaction mixture is stirred at a temperature in the region of 20° C. for 2 hours 30 minutes before being concentrated to dryness under reduced pressure. The resulting residue is taken up in 40 cm³ of dichloromethane in 10 cm³ of saturated aqueous sodium bicarbonate solution. Following phase separation, the aqueous phase is extracted with twice 15 cm³ of dichloromethane. The organic phases are combined, dried over magnesium sulphate, filtered and then concentrated to dryness under reduced pressure to give 811 mg of a crude product which is purified by flash chromatography on a column of 70 g of silica (Merck 15-40 μm; eluent: ethyl acetate 100). Concentration of the fractions under reduced pressure gives 470 mg of a yellow oil which, after trituration in pentane, filtration and drying under vacuum at a temperature in the region of 40° C., gives 184 mg of a pale yellow solid. This solid is again purified by flash chromatography on a column of 10 g of silica (Merck 15-40 μm; eluent: dichloromethane/methanol 98/2). Concentration of the fractions under reduced pressure gives 159 mg of a solid which is dried for 48 hours under vacuum at a temperature in the region of 40° C. to give 137 mg of 3-[{1-[bis(4-chlorophenyl)methyl]azetidin-3-yl}(methylsulphonyl)amino]-5-fluoro-N-(pyrimidin-2-yl)benzamide in the form of a pale yellow solid.

1H NMR spectrum (400 MHz; (δ in ppm); (DMSO-d6); referenced at 2.50 ppm): 2.77 (m, 2H); 3.03 (s, 3H); 3.37 (m, 2H); 4.41 (s, 1H); 4.73 (m, 1H); 7.26 (t, J=4.9 Hz, 1H); 7.31 (d, J=8.6 Hz, 4H); 7.36 (d, J=8.6 Hz, 4H); 7.47 (dt, J=9.5; 2.0 Hz, 1H); 7.67-7.83 (m, 2H); 8.73 (d, J=4.9 Hz, 2H); 11.17 (extended m, 1H)

Mass spectrum: ES m/z=600 [M+H]⁺; m/z=366 ([M+H−C₁₃H₈Cl₂]⁺, base peak); m/z=235 [C₁₃H₉Cl₂]⁺; m/z=598 [M−H]⁻

Elemental analysis:

Calculated: C: 56.01%-H: 4.03%-N: 11.66%-S: 5.34%

Measured: C: 55.26%-H: 4.03%-N: 11.50%-S: 5.22%-H₂O=0.85%

### Example 6

### 3-[{1-[Bis(4-chlorophenyl)methyl]azetidin-3-yl)(methylsulphonyl)amino]-5-fluoro-N-(5-methylisoxazol-3-yl)benzamide (Compound 21)

A solution of 400 mg of 3-[{1-[bis(4-chlorophenyl)methyl]azetidin-3-yl}-(methylsulphonyl)amino]-5-fluorobenzoic acid and 4 drops of dimethylformamide in 4 cm³ of dichloromethane is admixed with a solution of 245 μl of thionyl chloride in 1 cm³ of dichloromethane. The reaction mixture is stirred for 2 hours 20 minutes at a temperature in the region of 35° C., then cooled to a temperature in the region of 20° C., before being concentrated to dryness under reduced pressure after addition of a few cm³ of toluene. The resulting solid is dissolved in 10 cm³ of dichloromethane and 10 cm³ of tetrahydrofuran. This solution is admixed with a solution of 90 mg of 3-amino-5-methyloxazole in 2 cm³ of dichloromethane. The reaction mixture is stirred at a temperature in the region of 20° C. overnight before being concentrated to dryness under reduced pressure. The resulting residue is taken up in 50 cm³ of dichloromethane, 15 cm³ of water and 15 cm³ of saturated aqueous sodium bicarbonate solution. Following phase separation, the aqueous phase is extracted with 2 times 30 cm³ of dichloromethane. The organic phases are combined, washed with 15 cm³ of saturated aqueous sodium chloride solution, dried over sodium sulphate, filtered and then concentrated to dryness under reduced pressure to give 0.44 g of a foam. This crude product is purified by flash chromatography on a column of 30 g of silica (Merck 15-40 μm; eluent: heptane/ethyl acetate 60/40). Concentration of the fractions under reduced pressure and drying under vacuum at a temperature in the region of 40° C. give 240 mg of 3-[{1-[bis(4-chlorophenyl)methyl]azetidin-3-yl}(methyl sulphonyl)amino]-5-fluoro-N-(5-methylisoxazol-3-yl)benzamide in the form of a white solid.

m.p.: 222-224° C.

1H NMR spectrum (400 MHz; (δ in ppm); (DMSO-d6); referenced at 2.50 ppm): 2.42 (broad s, 3H); 2.76 (m, 2H); 3.02 (s, 3H); 3.38 (m, 2H); 4.41 (s, 1H); 4.72 (m, 1H); 6.75 (broad s, 1H); 7.31 (d, J=8.6 Hz, 4H); 7.36 (d, J=8.6 Hz, 4H); 7.49 (dt, J=9.3; 2.2 Hz, 1H); 7.79-7.85 (m, 2H); 11.45 (s, 1H)

Mass spectrum: ES m/z=603 [M+H]⁺; m/z=601 [M−H]⁻

Elemental analysis:

Calculated: C: 55.73%-H: 4.18%-N: 9.28%-S: 5.31%

Measured: C: 55.72%-H: 4.18%-N: 9.17%-S: 5.32%

Table 1 below illustrates the chemical structures (I) and the physical properties of some examples of compounds according to the invention. In this table:

R represents a methyl group;

R3 and R4 each represent a phenyl group substituted by a chlorine atom in para position.

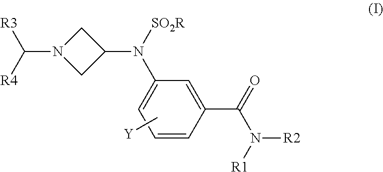

TABLE 1

| Compound | 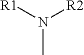 | Y | Characterizations |
|---|---|---|---|
| 1 | 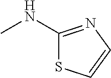 | H | m.p.: 244° C.; 1H NMR spectrum (400 MHz; (δ in ppm); (DMSO-d6); referenced at 2.50 ppm): 2.74 (m, 2H); 3.00 (s, 3H); 3.37 (m, 2H); 4.39 (s, 1H); 4.74 (m, 1H); 7.27 (masked m, 1H); 7.29 (d, J = 8.4 Hz, 4H); 7.35 (d, J = 8.4 Hz, 4H); 7.51-7.61 (m, 3H); 8.02-8.08 (m, 2H); 12.72 (extended m, 1H); mass spectrum: ES m/z = 587 (M + H)⁺; m/z = 585 (M − H)⁻; elemental analysis: calculated: C: 55.20%- H: 4.12%- N: 9.54%- S: 10.91%; measured: C: 53.99%- H: 4.10%- N: 9.02%- S: 10.12%- H₂O: 2.65% |
| 2 | 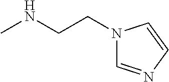 | H | 1H NMR spectrum (300 MHz; (δ in ppm); (DMSO-d6); referenced at 2.50 ppm): 2.69 (m, 2H); 2.96 (s, 3H); 3.31 (masked m, 2H); 3.58 (m, 2H); 4.16 (t, J = 6.1 Hz, 2H); 4.37 (s, 1H); 4.72 (m, 1H); 6.85 (broad s, 1H); 7.14 (broad s, 1H); 7.31 (d, J = 8.7 Hz, 4H); 7.36 (d, J = 8.7 Hz, 4H); 7.44-7.53 (m, 2H); 7.59 (broad s, 1H); 7.70 (broad s, 1H); 7.76 (m, 1H); 8.65 (broad t, J = 5.6 Hz, 1H); mass spectrum: ES m/z = 598 (M + H)⁺; m/z = 364 ([M + H—C₁₃H₈Cl₂], base peak); m/z = 596 [M − H]⁻; m/z = 642 ([M + HCO₂H—H]⁻, base peak) |
| 3 | 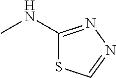 | H | 1H NMR spectrum (400 MHz; (δ in ppm); (DMSO-d6); referenced at 2.50 ppm): 2.74 (m, 2H); 3.01 (s, 3H); 3.37 (m, 2H); 4.39 (s, 1H); 4.74 (m, 1H); 7.30 (d, J = 8.6 Hz, 4H); 7.35 (d, J = 8.6 Hz, 4H); 7.59 (m, 2H); 8.01-8.11 (m, 2H); 9.21 (s, 1H); 13.18 (extended m, 1H); mass spectrum: ES m/z = 588 [M + H]⁺ elemental analysis: calculated: C: 53.06%- H: 3.94%- N: 11.90%- S: 10.90%; measured: C: 53.02- H: 4.20%- N: 11.59%- S: 10.41%- H₂O: 0.99% |
| 4 | 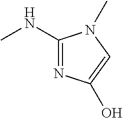 | H | m.p.: 134° C.; 1H NMR spectrum (400 MHz; (δ in ppm); (DMSO-d6); referenced at 2.50 ppm): 2.71 (m, 2H); 2.94 (s, 3H); 3.10 (s, 3H); 3.34 (partially masked m, 2H); 4.10 (s, 2H); 4.39 (s, 1H); 4.77 (m, 1H); 7.30 (d, J = 8.6 Hz, 4H); 7.35 (d, J = 8.6 Hz, 4H); 7.35 (m, 2H); 7.99 (broad s, 1H); 8.10 (m, 1H); 11.23 (extended m, 1H); mass spectrum: ES m/z = 600 [M + H]⁺; m/z = 598 [M − H]⁻ |
| 5 | 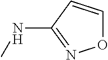 | H | m.p.: 218° C.; 1H NMR spectrum (400 MHz; (δ in ppm); (DMSO-d6); referenced at 2.50 ppm): 2.74 (m, 2H); 3.00 (s, 3H); 3.36 (m, 2H); 4.38 (s, 1H); 4.74 (m, 1H); 7.05 (d, J = 1.7 Hz, 1H); 7.29 (d, J = 8.8 Hz, 4H); 7.35 (d, J = 8.8 Hz, 4H); 7.56 (m, 2H); 7.95 (broad s, 1H); 7.99 (m, 1H); 8.86 (d, J = 1.7 Hz, 1H); 11.51 (s, 1H); mass spectrum: ES m/z = 571 ([M + H]⁺, base peak); m/z = 569 [M − H]⁻; elemental analysis: calculated: C: 56.75%- H: 4.23%- N: 9.80%- S: 5.61%; measured: C: 56.07%- H: 4.14%- N: 9.73%- S: 5.46%- H₂O: 1.31% |
| 6 | 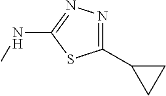 | H | m.p.: 226° C.; 1H NMR spectrum (400 MHz; (δ in ppm); (DMSO-d6); referenced at 2.50 ppm): 1.01 (m, 2H); 1.16 (m, 2H); 2.42 (m, 1H); 2.74 (m, 2H); 3.00 (s, 3H); 3.37 (m, 2H); 4.38 (s, 1H); 4.73 (m, 1H); 7.30 (d, J = 8.5 Hz, 4H); 7.35 (d, J = 8.5 Hz, 4H); 7.58 (m, 2H); 8.00-8.13 (m, 2H); 12.97 (extended m, 1H); mass spectrum: ES m/z = 628 ([M + H]⁺, base peak); m/z = 394 [M + H—C₁₃H₈Cl₂]; m/z = 626 [M − H]⁻; elemental analysis: calculated: C: 55.41%- H: 4.33%- N: 11.14%- S: 10.20%; measured: C: 55.50%- H: 4.36%- N: 11.16%- S: 9.94% |
| 7 | 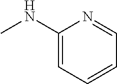 | H | m.p.: 202° C.; 1H NMR spectrum (400 MHz; (δ in ppm); (DMSO-d6); referenced at 2.50 ppm): 2.75 (m, 2H); 3.00 (s, 3H); 3.36 (m, 2H); 4.39 (s, 1H); 4.75 (m, 1H); 7.18 (m, 1H); 7.30 (d, J = 8.8 Hz, 4H); 7.35 (d, J = 8.8 Hz, 4H); 7.51-7.57 (m, 2H); 7.85 (m, 1H); 7.97 (s, 1H); 8.00 (m, 1H); 8.18 (d, J = 8.4 Hz, 1H); 8.40 (broad d, J = 5.0 Hz, 1H); 10.89 (broad s, 1H); mass spectrum: ES m/z = 581 [M + H]⁺; m/z = 347 [M + H—C₁₃H₈Cl₂]⁺; m/z = 579 [M − H]⁻; m/z = 1159 [2M − H]⁻ |
| 8 | 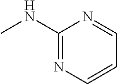 | H | m.p.: 137° C.; 1H NMR spectrum (400 MHz; (δ in ppm); (DMSO-d6); referenced at 2.50 ppm); all of the signals are broad, with: 2.74 (m, 2H); 3.00 (s, 3H); 3.35 (m, 2H); 4.39 (s, 1H); 4.74 (m, 1H); 7.22-7.39 (m, 9H); 7.54 (m, 2H); 7.88 (s, 1H); 7.93 (m, 1H); 8.73 (d, J = 4.9 Hz, 2H); 11.09 (s, 1H); mass spectrum: ES m/z = 582 [M + H]⁺; m/z = 235 ([C₁₃H₉Cl₂]⁺, base peak); m/z = 580 [M − H]⁻ |
| 9 | 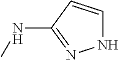 | H | m.p.: 150° C.; 1H NMR spectrum (400 MHz; (δ in ppm); (DMSO-d6); referenced at 2.50 ppm): 2.74 (m, 2H); 2.99 (s, 3H); 3.36 (m, 2H); 4.38 (s, 1H); 4.74 (m, 1H); 6.65 (broad m, 1H); 7.30 (d, J = 8.8 Hz, 4H); 7.35 (d, J = 8.8 Hz, 4H); 7.46-7.56 (m, 2H); 7.66 (broad m, 1H); 7.94 (broad s, 1H); 7.97 (m, 1H); 10.90 (broad m, 1H); 12.46 (broad m, 1H); mass spectrum: ES m/z = 570 ([M + H]⁺, base peak); m/z = 336 [M + H—C₁₃H₈Cl₂]⁺; m/z = 1139 [2M + H]⁺ |
| 10 | 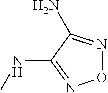 | H | m.p.: 255° C.; 1H NMR spectrum (400 MHz; (δ in ppm); (DMSO-d6); referenced at 2.50 ppm): 2.73 (m, 2H); 3.00 (s, 3H); 3.37 (m, 2H); 4.39 (s, 1H); 4.75 (m, 1H); 6.07 (broad s, 2H); 7.31 (d, J = 8.8 Hz, 4H); 7.36 (d, J = 8.8 Hz, 4H); 7.59 (m, 2H); 7.91 (broad s, 1H); 7.97 (m, 1H); 10.98 (broad s, 1H); mass spectrum: ES m/z = 587 ([M + H]⁺, base peak); m/z = 1173 [2M + H]⁺ |
| 11 | 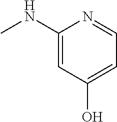 | H | m.p.: 208° C.; 1H NMR spectrum (400 MHz; (δ in ppm); (DMSO-d6); referenced at 2.50 ppm): 2.74 (m, 2H); 3.00 (s, 3H); 3.36 (m, 2H); 4.39 (s, 1H); 4.75 (m, 1H); 6.58 (broad d, J = 5.9 Hz, 1H); 7.30 (d, J = 8.6 Hz, 4H); 7.35 (d, J = 8.6 Hz, H); 7.49-7.57 (m, 2H); 7.71 (broad m, 1H); 7.90-8.00 (m, 2H); 8.07 (broad d, J = 5.9 Hz, 1H); 10.22-10.85 (broad m, 2H); mass spectrum: ES m/z = 597 ([M + H]⁺, base peak); m/z = 363 [M + H—C₁₃H₈Cl₂]⁺ |
| 12 | 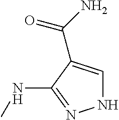 | H | m.p.: 174° C.; 1H NMR spectrum (400 MHz; (δ in ppm); (DMSO-d6); referenced at 2.50 ppm): 2.74 (m, 2H); 2.98 (s, 3H); 3.38 (m, 2H); 4.42 (s, 1H); 4.78 (m, 1H); 7.30 (d, J = 8.7 Hz, 4H); 7.36 (d, J = 8.7 Hz, 4H); 7.42 (extended m, 1H); 7.61 (extended m, 3H); 7.85 (extended m, 3H); 11.15 (extended m, 1H); 12.76 (extended m, 1H); mass spectrum: ES m/z = 613 [M + H]⁺; m/z = 379 [M + H—C₁₃H₈Cl₂]⁺; m/z = 611 [M − H]⁻ |
| 13 | 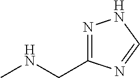 | H | m.p.: 210° C.; 1H NMR spectrum (400 MHz; (δ in ppm); (DMSO-d6); referenced at 2.50 ppm): 2.70 (m, 7H); 2.97 (s, 3H); 3.34 (partially masked m, 2H); 4.37 (s, 1H); 4.55 (d, J = 5.9 Hz, 2H); 4.73 (m, 1H); 7.31 (d, J = 8.8 Hz, 4H); 7.35 (d, J = 8.8 Hz, 4H); 7.46 -7.54 (m, 2H); 7.80 (broad s, 1H); 7.87 (m, 1H); 8.17 (extended m, 1H); 9.10 (broad t, J = 5.6 Hz, 1H); 13.80 (very extended m, 1H); mass spectrum: ES m/z = 585 [M + H]⁺; m/z = 583 [M − H]⁻ |
| 14 | 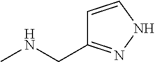 | H | m.p.: 224° C.; 1H NMR spectrum (400 MHz; (δ in ppm); (DMSO-d6); referenced at 2.50 ppm): 2.70 (m, 2H); 2.96 (s, 3H); 3.33 (m, 2H); 4.37 (s, 1H); 4.47 (extended m, 2H); 4.72 (m, 1H); 6.16 (broad s, 1H); 7.30 (d, J = 8.7 Hz, 4H); 7.35 (d, J = 8.7 Hz, 4H); 7.44-7.67 (m, 3H); 7.79 (broad s, 1H); 7.86 (m, 1H); 8.96 (extended m, 1H); 12.57 (extended m, 1H); mass spectrum: ES m/z = 584 ([M + H]⁺, base peak); m/z = 350 [M + H—C₁₃H₈Cl₂]⁺ |
| 15 | 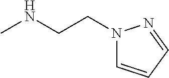 | H | m.p.: 264° C.; 1H NMR spectrum (400 MHz; (δ in ppm); (DMSO-d6); referenced at 2.50 ppm): 2.68 (m, 2H); 2.95 (s, 3H); 3.33 (partially masked m, 2H); 3.62 (m, 2H); 4.29 (t, J = 6.3 Hz, 2H); 4.37 (s, 1H); 4.71 (m, 1H); 6.17 (t, J = 2.0 Hz, 1H); 7.31 (d, J = 8.8 Hz, 4H); 7.36 (d, J = 8.8 Hz, 4H); 7.41 (broad d, J = 2.0 Hz, 1H); 7.44-7.52 (m, 2H); 7.67 (d, J = 2.0 Hz, 1H); 7.70 (broad s, 1H); 7.76 (m, 1H); 8.61 (t, J = 5.7 Hz, 1H); mass spectrum: ES m/z = 598 [M + H]⁺; m/z = 364 [M + H—C₁₃H₈Cl₂]⁺; m/z = 596 [M − H]⁻; m/z = 642 ([M + HCO₂H—H]⁻, base peak) |
| 16 | 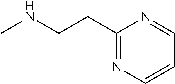 | H | m.p.: 120° C.; 1H NMR spectrum (400 MHz; (δ in ppm); (DMSO-d6); referenced at 2.50 ppm): 2.69 (m, 2H); 2.95 (s, 3H); 3.14 (t, J = 7.3 Hz, 2H); 3.33 (m, 2H); 3.71 (m, 2H); 4.37 (s, 1H); 4.72 (m, 1H); 7.28-7.39 (m, 9H); 7.41-7.54 (m, 2H); 7.72 (broad s, 1H); 7.77 (m, 1H); 8.63 (broad t, J = 5.6 Hz, 1H); 8.71 (d, J = 4.9 Hz, 2H); mass spectrum: ES m/z = 610 ([M + H]⁺, base peak); m/z = 376 [M + H—C₁₃H₈Cl₂]⁺; elemental analysis: calculated: C: 59.02%- H: 4.79%- N: 11.47%- S: 5.25%; measured: C: 59.04%- H: 5.33%- N: 10.70%- S: 4.77%- H₂O: 1.10% |
| 17 | 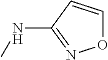 | F | m.p.: 219-221° C.; 1H NMR spectrum (400 MHz; (δ in ppm); (DMSO-d6); referenced at 2.50 ppm): 2.76 (m, 2H); 3.03 (s, 3H); 3.39 (m, 2H); 4.41 (s, 1H); 4.73 (m, 1H); 7.05 (d, J = 1.7 Hz, 1H); 7.31 (d, J = 8.6 Hz, 4H); 7.36 (d, J = 8.6 Hz, 4H); 7.50 (dt, J = 9.5; 2.0 Hz, 1H); 7.80-7.85 (m, 2H); 8.88 (d, J = 1.7 Hz, 1H); 11.60 (s, 1H); mass spectrum: ES m/z = 589 [M + H]⁺; m/z = 587 [M − H]⁻; elemental analysis: calculated: C: 55.02%- H: 3.93%- N: 9.50%- S: 5.44%; measured: C: 55.21%- H: 4.04%- N: 9.30%- S: 4.96% |
| 18 | 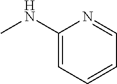 | F | 1H NMR spectrum (400 MHz; (δ in ppm); (DMSO-d6); referenced at 2.50 ppm): 2.77 (m, 2H); 3.03 (s, 3H); 3.39 (m, 2H); 4.42 (s, 1H); 4.74 (m, 1H); 7.20 (broad dd, J = 7.2; 4.9 Hz, 1H); 7.31 (d, J = 8.6 Hz, 4H); 7.37 (d, J = 8.6 Hz, 4H); 7.46 (dt, J = 9.5; 2.0 Hz, 1H); 7.78-7.90 (m, 3H); 8.17 (d, J = 8.3 Hz, 1H); 8.41 (broad d, J = 4.9 Hz, 1H); 10.99 (s, 1H); mass spectrum: ES m/z = 599 [M + H]⁺; m/z = 365 ([M + H—C₁₃H₈Cl₂]⁺; base peak); m/z = 597 [M − H]⁻; elemental analysis: calculated: C: 58.10%- H: 4.20%- N: 9.35%- S: 5.35%; measured: C: 57.79%- H: 4.37%- N: 9.26%- S: 4.98% |
| 19 | 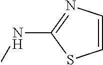 | F | 1H NMR spectrum (400 MHz; (δ in ppm); (DMSO-d6); referenced at 2.50 ppm): 2.77 (m, 2H); 3.03 (s, 3H); 3.40 (m, 2H); 4.41 (s, 1H); 4.73 (m, 1H); 7.27-7.33 (m, 5H); 7.36 (d, J = 8.6 Hz, 4H); 7.49 (dt, J = 9.3; 2.2 Hz, 1H); 7.58 (d, J = 3.7 Hz, 1H); 7.85-7.93 (m, 2H); 12.80 (extended m, 1H); mass spectrum: ES m/z = 605 [M + H]⁺; m/z = 371 ([M + H—C₁₃H₈Cl₂]⁺; m/z = 235 [(C₁₃H₉Cl₂]⁺, base peak); m/z = 603 [M − H]⁻ |
| 20 | 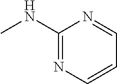 | F | 1H NMR spectrum (400 MHz; (δ in ppm); (DMSO-d6); referenced at 2.50 ppm): 2.77 (m, 2H); 3.03 (s, 3H); 3.37 (m, 2H); 4.41 (s, 1H); 4.73 (m, 1H); 7.26 (t, J = 4.9 Hz, 1H); 7.31 (d, J = 8.6 Hz, 4H); 7.36 (d, J = 8.6 Hz, 4H); 7.47 (dt, J = 9.5; 2.0 Hz, 1H); 7.67-7.83 (m, 2H); 8.73 (d, J = 4.9 Hz, 2H); 11.17 (extended m, 1H); mass spectrum: ES m/z = 600 [M + H]⁺; m/z = 366 ([M + H—C₁₃H₈Cl₂]⁺, base peak); m/z = 235 [C₁₃H₉Cl₂]⁺; m/z = 598 [M − H]⁻; elemental analysis: calculated: C: 56.01%- H: 4.03%- N: 11.66%- S: 5.34%; measured: C: 55.26%- H: 4.03%- N: 11.50%- S: 5.22%- H₂O: 0.85% |
| 21 | 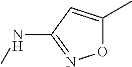 | F | m.p.: 222-224° C.; 1H NMR spectrum (400 MHz; (δ in ppm); (DMSO-d6); referenced at 2.50 ppm): 2.42 (broad s, 3H); 2.76 (m, 2H); 3.02 (s, 3H); 3.38 (m, 2H); 4.41 (s, 1H); 4.72 (m, 1H); 6.75 (broad s, 1H); 7.31 (d, J = 8.6 Hz, 4H); 7.36 (d, J = 8.6 Hz, 4H); 7.49 (dt, J = 9.3; 2.2 Hz, 1H); 7.79-7.85 (m, 2H); 11.45 (s, 1H); mass spectrum: ES m/z = 603 [M + H]⁺; m/z = 601 [M − H]⁻; elemental analysis: calculated: C: 55.73%- H: 4.18%- N: 9.28%- S: 5.31%; measured: C: 55.72%- H: 4.18%- N: 9.17%- S: 5.32% |
| 22 | 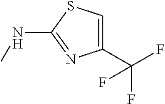 | F | 1H NMR spectrum (400 MHz; (δ in ppm); (DMSO-d6); referenced at 2.50 ppm): 2.77 (m, 2H); 3.04 (s, 3H); 3.40 (m, 2H); 4.41 (s, 1H); 4.73 (m, 1H); 7.30 (d, J = 8.7 Hz, 4H); 7.36 (d, J = 8.7 Hz, 4H); 7.52 (broad d, J = 9.2 Hz, 1H); 7.87-7.95 (m, 2H); 8.04 (broad m, 1H); 13.21 (s, 1H); mass spectrum: ES m/z = 673 [M + H]⁺; m/z = 671 [M − H]⁻ |
| 23 | 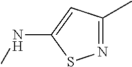 | F | 1H NMR spectrum (400 MHz; (δ in ppm); (DMSO-d6); referenced at 2.50 ppm): 2.37 (s, 3H); 2.76 (m, 2H); 3.03 (s, 3H); 3.39 (m, 2H); 4.41 (s, 1H); 4.76 (m, 1H); 6.94 (s, 1H); 7.31 (d, J = 8.6 Hz, 4H); 7.36 (d, J = 8.6 Hz, 4H); 7.55 (dt, J = 9.6; 2.0 Hz, 1H); 7.84 (t, J = 2.0 Hz, 1H); 7.87 (dt, J = 9.0; 2.0 Hz, 1H); 12.30 (broad s, 1H); mass spectrum: ES m/z = 619 [M + H]⁺; m/z = 385 ([M + H—C₁₃H₈Cl₂]⁺, base peak); m/z = 235 [C₁₃H₉Cl₂]⁺; m/z = 617 [M − H]⁻ |
| 24 | 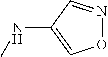 | F | m.p.: 234-236° C.; 1H NMR spectrum (400 MHz; (δ in ppm); (DMSO-d6); referenced at 2.50 ppm): 2.75 (m, 2H); 3.02 (s, 3H); 3.34-3.41 (m, 2H); 4.40 (s, 1H); 4.75 (m, 1H); 7.31 (d, J = 8.7 Hz, 4H); 7.37 (d, J = 8.7 Hz, 4H); 7.52 (dt, J = 9.3; 2.2 Hz, 1H); 7.74-7.80 (m, 2H); 8.75 (s, 1H); 9.27 (s, 1H); 10.81 (broad s, 1H); mass spectrum: ES m/z = 589 ([M + H]⁺, base peak); m/z = 235 [C₁₃H₉Cl₂]⁺; m/z = 587 ([M − H]⁻, base peak); m/z = 1175 [2M − H]⁻; elemental analysis: calculated: C: 55.02%- H: 3.93%- N: 9.50%- S: 5.44%; measured: C: 54.72%- H: 4.06%- N: 9.39%- S: 5.24% |
| 25 | 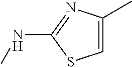 | F | 1H NMR spectrum (400 MHz; (δ in ppm); (DMSO-d6); referenced at 2.50 ppm): 2.31 (s, 3H); 2.77 (m, 2H); 3.03 (s, 3H); 3.39 (m, 2H); 4.41 (s, 1H); 4.72 (m, 1H); 6.83 (s, 1H); 7.30 (d, J = 8.8 Hz, 4H); 7.36 (d, J = 8.8 Hz, 4H); 7.47 (dt, J = 9.5; 2.0 Hz, 1H); 7.82-7.94 (m, 2H); 12.74 (extended m, 1H); mass spectrum: ES m/z = 619 [M + H]⁺; m/z = 385 ([M + H—C₁₃H₈Cl₂]⁺, base peak); m/z= 235 [C₁₃H₉Cl₂]⁺; m/z = 617 ([M − H]⁻, base peak); |
| 26 | 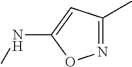 | F | m.p.: 224-226° C.; 1H NMR spectrum (400 MHz; (δ in ppm); (DMSO-d6); referenced at 2.50 ppm): 2.22 (s, 3H); 2.76 (m, 2H); 3.02 (s, 3H); 3.38 (m, 2H); 4.41 (s, 1H); 4.73 (m, 1H); 6.32 (s, 1H); 7.31 (d, J = 8.6 Hz, 4H); 7.36 (d, J = 8.6 Hz, 4H); 7.50 (dt, J = 9.5; 2.2 Hz, 1H); 7.73-7.87 (m, 2H); 12.03 (m, 1H); mass spectrum: ES m/z = 603 ([M + H]⁺, base peak); m/z = 235 [C₁₃H₉Cl₂]⁺; m/z = 601 [M − H]⁻; elemental analysis: calculated: C: 55.73%- H: 4.18%- N: 9.28%- S: 5.31%; measured: C: 55.56%- H: 4.17%- N: 9.10%- S: 4.75% |
| 27 | 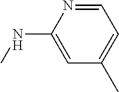 | F | m.p.: 184-186° C.; 1H NMR spectrum (400 MHz; (δ in ppm); (DMSO-d6); referenced at 2.50 ppm): 2.36 (s, 3H); 2.78 (m, 2H); 3.03 (s, 3H); 3.38 (m, 2H); 4.42 (s, 1H); 4.74 (m, 1H); 7.04 (dd, J = 4.9; 1.5 Hz, 1H); 7.31 (d, J = 8.6 Hz, 4H); 7.37 (d, J = 8.6 Hz, 4H); 7.46 (dt, J = 9.3; 2.2 Hz, 1H); 7.77-7.86 (m, 2H); 8.02 (broad s, 1H); 8.26 (d, J = 4.9 Hz, 1H); 10.90 (s, 1H); mass spectrum: ES m/z = 613 [M + H]⁺; m/z = 379 ([M + H—C₁₃H₈Cl₂]⁺, base peak); m/z = 235 [C₁₃H₉Cl₂]⁺; m/z = 611 [M − H]⁻; elemental analysis: calculated: C: 58.73%- H: 4.44%- N: 9.13%- S: 5.23%; measured: C: 58.99%- H: 4.66%- N: 8.77%- S: 4.85% |
| 28 | 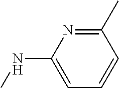 | F | m.p.: 176-178° C.; 1H NMR spectrum (400 MHz; (δ in ppm); (DMSO-d6); referenced at 2.50 ppm): 2.46 (s, 3H); 2.78 (m, 2H); 3.03 (s, 3H); 3.38 (m, 2H); 4.42 (s, 1H); 4.74 (m, 1H); 7.05 (d, J = 8.0 Hz, 1H); 7.30 (d, J = 8.7 Hz, 4H); 7.37 (d, J = 8.7 Hz, 4H); 7.45 (dd, J = 9.3; 2.0 Hz, 1H); 7.74 (t, J = 8.0 Hz, 1H); 7.79-7.88 (m, 2H); 7.99 (d, J = 8.0 Hz, 1H); 10.92 (s, 1H); mass spectrum: ES m/z = 613 [M + H]⁺; m/z = 379 ([M + H—C₁₃H₈Cl₂]⁺, base peak); m/z = 235 [C₁₃H₉Cl₂]⁺; m/z = 611 [M − H]⁻; elemental analysis: calculated: C: 58.73%- H: 4.44%- N: 9.13%- S: 5.23%; measured: C: 58.48%- H: 4.83%- N: 8.75%- S: 4.88%- H₂O: 2.65% |
| 29 | 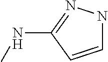 | F | 1H NMR ssectrum (400 MHz; (δ in ppm); (DMSO-d6); referenced at 2.50 ppm): 2.77 (m, 2H); 3.02 (s, 3H); 3.38 (m, 2H); 4.41 (s, 1H); 4.73 (m, 1H); 6.65 (broad s, 1H); 7.31 (d, J = 8.6 Hz, 4H); 7.36 (d, J = 8.6 Hz, 4H); 7.43 (dt, J = 9.5; 2.0 Hz, 1H); 7.67 (broad s, 1H); 7.76-7.95 (m, 2H); 11.00 (broad s, 1H); 12.49 (broad s, 1H); mass spectrum: ES m/z = 588 [M + H]⁺; m/z = 354 [M + H—C₁₃H₈Cl₂]⁺; m/z = 235 ([C₁₃H₉Cl₂]⁺, base peak); m/z = 586 [M − H]⁻ |
| 30 | 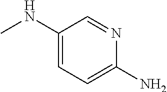 | H | m.p.: 210° C.; 1H NMR spectrum (400 MHz; (δ in ppm); (DMSO-d6); referenced at 2.50 ppm): 2.73 (m, 2H); 2.98 (s, 3H); 3.35 (m, 2H); 4.38 (s, 1H); 4.75 (m, 1H); 5.78 (s, 2H); 6.46 (d, J = 8.8 Hz, 1H); 7.30 (d, J = 8.8 Hz, 4H); 7.36 (d, J = 8.8 Hz, 4H); 7.44-7.60 (m, 2H); 7.66 (dd, J = 8.8; 2.6 Hz, 1H); 7.85 (t, J = 1.5 Hz, 1H); 7.92 (dt, J = 7.3; 1.5 Hz, 1H); 8.18 (d, J = 2.6 Hz, 1H); 9.99 (s, 1H); mass spectrum: ES m/z = 596 [M + H]⁺; m/z = 362 [M + H—C₁₃H₈Cl₂]⁺; m/z = 235 ([C₁₃H₉Cl₂]⁺, base peak) |
| 31 | 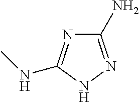 | H | m.p.: 222° C.; 1H NMR spectrum (400 MHz; (δ in ppm); (DMSO-d6); referenced at 2.50 ppm): 2.72 (m, 2H); 2.97 (s, 3H); 3.35 (m, 2H); 4.40 (s, 1H); 4.73 (m, 1H); 5.65 (broad s, 2H); 7.30 (d, J = 8.8 Hz, 4H); 7.36 (d, J = 8.8 Hz, 4H); 7.48-7.55 (m, 2H); 7.62 (broad s, 2H); 7.93 (broad s, 1H); 8.04 (m, 1H); mass spectrum: ES m/z = 586 [M + H]⁺; m/z = 352 [M + H—C₁₃H₈Cl₂]⁺; m/z = 235 ([C₁₃H₉Cl₂]⁺, base peak); elemental analysis: calculated: C: 53.25%- H: 4.30%- N: 16.72%- S: 5.47%; measured: C: 53.33%- H: 4.34%- N: 16.63%- S: 5.26% |
| 32 | 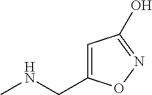 | H | m.p.: 209° C.; 1H NMR spectrum (300 MHz; (δ in ppm); (DMSO-d6); referenced at 2.50 ppm): 2.71 (m, 2H); 2.96 (s, 3H); 3.33 (masked m, 2H); 4.37 (s, 1H); 4.45 (d, J = 6.3 Hz, 2H); 4.73 (m, 1H); 5.85 (s, 1H); 7.30 (d, J = 8.8 Hz, 4H); 7.35 (d, J = 8.8 Hz, 4H); 7.42-7.59 (m, 2H); 7.79 (broad s, 1H); 7.85 (m, 1H); 9.13 (t, J = 6.3 Hz, 1H); mass spectrum: ES m/z = 601 [M + H]⁺ |
| 33 | 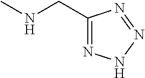 | H | m.p.: 124° C.; 1H NMR spectrum (400 MHz; (δ in ppm); (DMSO-d6); referenced at 2.50 ppm): 2.70 (m, 2H); 2.96 (s, 3H); 3.33 (partially masked m, 2H); 4.37 (s, 1H); 4.71 (d, J = 5.6 Hz, 2H); 4.74 (m, 1H); 7.31 (d, J = 8.4 Hz, 4H); 7.35 (d, J = 8.4 Hz, 4H); 7.45-7.56 (m, 2H); 7.80 (broad s, 1H); 7.86 (m, 1H); 9.15 (t, J = 5.6 Hz, 1H); mass spectrum: ES m/z = 586 [M + H]⁺; m/z = 584 [M − H]⁻ |
| 34 | 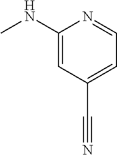 | F | m.p.: 165° C.; 1H NMR spectrum (400 MHz, δ in ppm, DMSO-d6): 2.77 (m, 2 H); 3.03 (s, 3 H); 3.38 (m, 2 H); 4.42 (s, 1H); 4.74 (quin, J = 6.6 Hz, 1 H); 7.29 to 7.33 (m, 4 H); 7.34 to 7.41 (m, 4 H); 7.49 (d, J = 9.3 Hz, 1 H); 7.64 (d, J = 4.9 Hz, 1 H); 7.79 to 7.90 (m, 2 H); 8.48 (s, 1 H); 8.66 (d, J = 4.9 Hz, 1 H); 11.43 (broad s, 1 H) mass spectrum: ES [M + H]+: m/z 624; [M − H]−: m/z 622 |
| 35 | 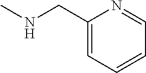 | F | 1H NMR spectrum (400 MHz, δ in ppm, DMSO-d6): 2.71 to 2.77 (m, 2 H); 3.01 (s, 3 H); 3.33 to 3.39 (m, 2H); 4.40 (s, 1 H); 4.57 (d, J = 6.0 HZ, 2 H); 4.72 (dq, J = 6.7 and 6.8 Hz, 1H); 7.25 to 7.38 (m, 9 H); 7.44 (dt, J = 2.2 and 9.5 Hz, 1 H); 7.68 to 7.78 (m, 3 H); 8.50 (d, J = 3.9 Hz, 1 H); 9.25 (t, J = 6.0 Hz, 2 H) mass spectrum: ES [M + H]+: m/z 613; [M − H]−: m/z 611 |
| 36 | 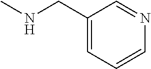 | F | 1H NMR spectrum (400 MHz, δ in ppm, DMSO-d6): 2.69 to 2.76 (m, 2 H); 2.99 (s, 3 H); 3.33 to 3.43 (m, 2H); 4.40 (s, 1 H); 4.50 (d, J = 5.8 Hz, 2 H); 4.72 (quin, J = 6.6 Hz, 1 H); 7.28 to 7.38 (m, 9 H); 7.44 (dt, J = 1.9 and 9.6 Hz, 1 H); 7.64 to 7.75 (m, 3 H); 8.47 (dd, J = 1.8 and 4.8 Hz, 1 H); 8.56 (d, J = 1.3 Hz, 1 H); 9.22 (t, J = 5.8 Hz, 1 H) mass spectrum: ES [M + H]+: m/z 613; [M − H]−: m/z 611 |
| 37 | 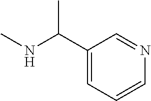 | F | 1H NMR spectrum (400 MHz, δ in ppm, DMSO-d6): 1.52 (d, J = 7.3 Hz, 3 H); 2.68 to 2.76 (m, 2 H); 2.99 (s, 3 H); 3.37 (masked m, 2 H); 4.40 (s, 1 H); 4.73 (quin, J = 6.7 Hz, 1 H); 5.18 (q, 7.3 Hz, 1 H); 7.29 to 7.38 (m, 9 H); 7.45 (dt, J = 2.2 and 9.3 Hz, 1 H); 7.68 (s, 1 H); 7.73 to 7.81 (m, 2 H); 8.46 (dd, J = 1.7 and 4.9 Hz, 1 H); 8.61 (d, J = 2.2 Hz, 1 H); 9.02 (d, J = 7.6 Hz, 1 H) mass spectrum: ES [M + H]+: m/z 627; [M − H]−: m/z 625 |
| 38 | 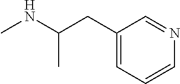 | F | 1H NMR spectrum (400 MHz, δ in ppm, DMSO-d6): 1.19 (d, J = 6.6 Hz, 3 H); 2.65 to 2.74 (m, 2 H); 2.83 (d, J = 7.1 Hz, 2 H); 2.99 (s, 3 H); 3.28 to 3.32 (masked m, 2 H); 4.17 to 4.28 (m, 1 H); 4.61 (s, 1 H); 4.72 (q, J = 6.6 Hz, 1 H); 7.15 to 7.25 (m, 1 H); 7.27 to 7.45 (m, 9 H); 7.50 to 7.65 (m, 3 H); 8.34 (dd, J = 1.7 and 4.6 Hz, 1 H); 8.40 to 8.50 (m, J = 2.4 Hz, 2 H) mass spectrum: ES [M + H]+: m/z 641; [M − H]−: m/z 639 |
| 39 | 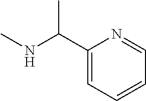 | F | 1H NMR spectrum (400 MHz, δ in ppm, DMSO-d6): 1.52 (d, J = 7.1 Hz, 3 H); 2.70 to 2.78 (m, 2 H); 2.99 (s, 3 H); 3.30 to 3.33 (masked m, 2 H); 4.40 (s, 1 H); 4.72 (, J = 6.2 and 6.5 and 6.6 Hz, 1 H); 5.19 (qd, J = 7.1 and 7.3 Hz, 1 H); 7.20 to 7.48 (m, 11 H); 7.60 to 7.84 (m, 3 H); 8.51 (d, J = 4.6 Hz, 1 H); 8.96 (d, J = 7.8 Hz, 1 H) mass spectrum: ES [M + H]+: m/z 627; [M − H]−: m/z 625 |
| 40 | 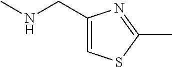 | F | 1H NMR spectrum (400 MHz, δ in ppm, DMSO-d6): 2.62 (s, 3 H); 2.70 to 2.77 (m, 2 H); 3.00 (s, 3 H); 3.32 to 3.39 (m, 2 H); 4.40 (s, 1 H); 4.51 (d, J = 5.9 Hz, 2 H); 4.72 (quin, J = 6.7 Hz, 1 H); 7.24 (s, 1 H); 7.28 to 7.38 (m, 8 H); 7.42 (dt, J = 2.3 and 9.4 Hz, 1 H); 7.66 to 7.77 (m, 2 H); 9.15 (t, J = 6.0 Hz, 1 H) mass spectrum: ES [M + H]+: m/z 633; [M −H]−: m/z 631 |
| 41 | 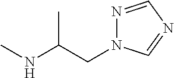 | F | 1H NMR spectrum (400 MHz, δ in ppm, DMSO-d6): 1.16 (d, J = 6.6 Hz, 3 H); 2.72 (q, J = 5.5 Hz, 2 H); 2.99 (s, 3 H); 3.32 to 3.45 (m, 2 H); 4.23 to 4.48 (m, 4 H); 4.71 (quin, J = 6.8 Hz, 1 H); 7.29 to 7.39 (m, 8 H); 7.41 (dt, J = 2.4 and 9.3 Hz, 1 H); 7.53 to 7.63 (m, J = 1.0 Hz, 2H); 7.89 (s, 1 H); 8.41 to 8.50 (m, 2 H) mass spectrum: ES [M + H]+: m/z 631; [M − H]−: m/z 629 |
| 42 | 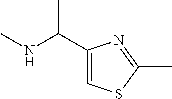 | F | 1H NMR spectrum (400 MHz, δ in ppm, DMSO-d6): 1.51 (d, J = 7.1 Hz, 3 H); 2.62 (s, 3 H); 2.74 (q, J = 6.9 Hz, 2 H); 3.00 (s, 3 H); 3.31 to 3.40 (m, 2 H); 4.40 (s, 1 H); 4.72 (quin, J = 6.7 Hz, 1 H); 5.25 (quin, J = 7.3 Hz, 1 H); 7.24 (s, 1 H); 7.27 to 7.39 (m, 8 H); 7.42 (dt, J = 2.2 and 9.5 Hz, 1 H); 7.69 (s, 1 H); 7.76 (d, J = 8.8 Hz, 1H); 8.91 (d, J = 7.8 Hz, 1 H) mass spectrum: ES [M + H]+: m/z 647; [M − H]−: m/z 645 |
| 43 | 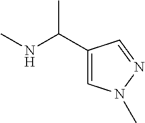 | F | 1H NMR spectrum (400 MHz, δ in ppm, DMSO-d6): 1.45 (d, J = 6.8 Hz, 3 H); 2.69 to 2.76 (m, 2 H); 2.99 (s, 3 H); 3.32 to 3.38 (m, 2 H); 3.78 (s, 3 H); 4.40 (s, 1 H); 4.65 to 4.77 (m, 1 H); 5.13 (quin, J = 7.3 Hz, 1 H); 7.27 to 7.49 (m, 10 H); 7.60 (s, 1 H); 7.67 (s, 1 H); 7.71 (d, J = 9.3 Hz, 1 H); 8.72 (d, J = 8.1 Hz, 1 H) mass spectrum: ES [M + H]+: m/z 630; [M − H]−: m/z 628 |
| 44 | 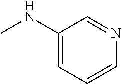 | F | 1H NMR spectrum (400 MHz, δ in ppm, DMSO-d6): 2.73 to 2.79 (m, 2 H); 3.03 (s, 3 H); 3.35 to 3.42 (m, 2H); 4.42 (s, 1 H); 4.76 (dq, J = 6.7 and 7.0 Hz, 1 H); 7.29 to 7.39 (m, 8 H); 7.42 (dd, J = 4.6 and 8.3 Hz, 1 H); 7.51 (dt, J = 2.2 and 9.5 Hz, 1 H); 7.77 (s, 1 H); 7.79 to 7.85 (m, 1 H); 8.11 to 8.17 (m, 1 H); 8.32 to 8.36 (m, 1 H); 8.90 (d, J = 2.7 Hz, 1 H); 10.49 (s, 1 H) mass spectrum: ES [M + H]+: m/z 599; [M − H]−: m/z 597 |
| 45 | 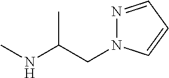 | F | 1H NMR spectrum (400 MHz, δ in ppm, DMSO-d6): 1.12 d, J = 6.8 Hz, 3 H); 2.69 to 2.77 (m, 2 H); 2.99 (s, 3 H); 3.32 to 3.40 (m, 2 H); 4.15 to 4.30 (m, 2 H); 4.31 to 4.44 (m, 2 H); 4.71 (, J = 6.7 and 6.8 and 7.1 Hz, 1 H); 6.13 (t, J = 2.1 Hz, 1 H); 7.24 to 7.46 (m, 10 H); 7.53 to 7.62 (m, 2 H); 7.66 (d, J = 2.2 Hz, 1 H); 8.43 (d, J = 8.1 Hz, 1 H) mass spectrum: ES [M + H]+: m/z 630; [M − H]−: m/z 628 |
| 46 | 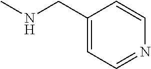 | F | 1H NMR spectrum (400 MHz, δ in ppm, DMSO-d6): 2.70 to 2.78 (m, J = 7.6 and 7.6 Hz, 2 H); 3.00 (s, 3 H); 3.33 to 3.40 (m, 2 H); 4.40 (s, 1 H); 4.50 (d, J = 5.9 Hz, 2 H); 4.72 (quin, J = 6.6 Hz, 1 H); 7.25 to 7.39 (m, 10H); 7.45 (ddd, J = 2.2 and 2.3 and 9.4 Hz, 1 H); 7.64 to 7.75 (m, 2 H); 8.50 (d, J = 5.9 Hz, 2 H); 9.25 (t, J = 6.1 Hz, 1 H) mass spectrum: ES [M + H]+: m/z 613; [M − H]−: m/z 611 |
| 47 | 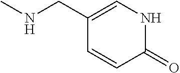 | F | 1H NMR spectrum (400 MHz, δ in ppm, DMSO-d6): 2.68 to 2.75 (m, 2 H); 2.98 (s, 3 H); 3.32 to 3.38 (m, 2H); 4.19 (d, J = 5.6 Hz, 2 H); 4.39 (s, 1 H); 4.71 (quin, J = 6.7 Hz, 1 H); 6.30 (d, J = 9.5 Hz, 1 H); 7.26 to 7.38 (m, 9 H); 7.42 (dd, J = 2.6 and 9.4 Hz, 2 H); 7.61 to 7.69 (m, 2 H); 8.97 (t, J = 5.7 Hz, 1 H); 11.44 (broad s, 1 H) mass spectrum: ES [M + H]+: m/z 629; [M − H]−: m/z 627 |
| 48 | 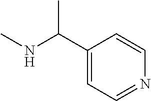 | F | 1H NMR spectrum (400 MHz, δ in ppm, DMSO-d6): 1.49 (d, J = 7.1 Hz, 3 H); 2.69 to 2.79 (m, 2 H); 2.99 (s, 3 H); 3.33 to 3.38 (m, 2 H); 4.40 (s, 1 H); 4.73 (quin, J = 6.7 Hz, 1 H); 5.13 (qd, J = 6.8 and 7.1 Hz, 1 H); 7.25 to 7.40 (m, 10 H); 7.45 (d, J = 8.6 Hz, 1 H); 7.69 (s, 1 H); 7.77 (d, J = 8.4 Hz, 1 H); 8.44 to 8.53 (m, 2 H); 8.99 (d, J = 7.6 Hz, 1 H) mass spectrum: ES [M + H]+: m/z 627; [M − H]−: m/z 625 |
| 49 | 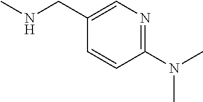 | F | 1H NMR spectrum (400 MHz, δ in ppm, DMSO-d6): 2.69 to 2.75 (m, 2 H); 2.99 (s, 6 H); 3.29 (s, 3 H); 3.32 to 3.38 (m, 2 H); 4.32 (d, J = 5.6 Hz, 2 H); 4.39 (s, 1 H); 4.71 (dq, J = 6.7 and 7.0 Hz, 1 H); 6.59 (d, J = 8.8 Hz, 1H); 7.33 (d, J = 10.3 Hz, 8 H); 7.41 (dt, J = 2.3 and 9.4 Hz, 1 H); 7.47 (dd, J = 2.4 and 8.8 Hz, 1 H); 7.60 to 7.71 (m, J = 0.7 and 1.5 Hz, 2 H); 8.06 (d, J = 1.7 Hz, 1 H); 9.02 (t, J = 5.6 Hz, 1 H) mass spectrum: ES [M + H]+: m/z 656; [M − H]−: m/z 654 |
| 50 | 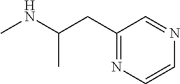 | F | 1H NMR spectrum (400 MHz, δ in ppm, DMSO-d6): 1.21 (d, J = 6.6 Hz, 3 H); 2.65 to 2.76 (m, 2 H); 2.93 to 3.11 (m, 5 H); 3.30 to 3.39 (m, 2 H); 4.34 to 4.50 (m, 2 H); 4.71 (quin, J = 6.7 Hz, 1 H); 7.26 to 7.44 (m, 9 H); 7.52 to 7.61 (m, 2 H); 8.41 (d, J = 2.7 Hz, 1 H); 8.43 to 8.50 (m, 2 H); 8.54 (d, J = 1.5 Hz, 1 H) mass spectrum: ES [M + H]+; m/z 642; [M − H]−: m/z 640 |
| 51 | 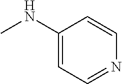 | F | 1H NMR spectrum (400 MHz, δ in ppm, DMSO-d6): 2.71 to 2.80 (m, 2 H); 3.03 (s, 3 H); 3.35 to 3.41 (m, 2H); 4.42 (s, 1 H); 4.76 (quin, J = 6.7 Hz, 1 H); 7.28 to 7.40 (m, 8 H); 7.52 (dt, J = 2.2 and 9.6 Hz, 1 H); 7.72 to 7.77 (m, 3 H); 7.79 to 7.85 (m, 1 H); 8.44 to 8.55 (m, 2 H); 10.61 (s, 1 H) mass spectrum: ES [M + H]+: m/z 599; [M − H]−: m/z 597 |
| 52 | 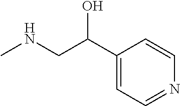 | F | 1H NMR spectrum (400 MHz, δ in ppm, DMSO-d6): 2.69 to 2.76 (m, J = 7.6 and 7.6 Hz, 2 H); 2.99 (s, 3 H); 3.33 to 3.52 (m, 4 H); 4.40 (s, 1 H); 4.67 to 4.81 (m, 2 H); 5.76 (d, J = 4.6 Hz, 1 H); 7.28 to 7.44 (m, 11 H); 7.58 to 7.66 (m, J = 2.2 Hz, 2 H); 8.47 to 8.51 (m, 2 H); 8.74 (t, J = 5.5 Hz, 1 H) mass spectrum: ES [M + H]+: m/z 643; [M − H]−: m/z 641 |

The compounds according to the invention were the subject of pharmacological tests for determining the activity with respect to human cannabinoid CB1-type receptors. The efficacy of the compounds of formula (I) was determined in a functional test which measures cannabinoid CB1 receptor activity (intracellular cyclic AMP test). The test for detecting intracellular cyclic AMP in U373MG cells naturally expressing the human CB1 receptor was carried out as described in the following reference: Bouaboula et al., 1995, J. Biol. Chem. 270:13973-13980. The HTRF cAMP Dynamic Kit from CisBio was used to quantify the intracellular cyclic AMP. In this test, the IC₅₀ values are between 0.001 μM and 2 μM.

For example, compounds 5, 10, 11, 15, 18, 34, 39 and 47 showed IC₅₀ values of, respectively, 0.006; 0.04; 0.170; 0.134; 0.006, 0.02, 0.033 and 0.009 μM.

Other tests consisting in measuring the in vivo activity of the compounds of the invention were carried out. The antagonist activity of said compounds was shown by means of the model of hypothermia induced by a cannabinoid CB1 receptor agonist (racemic CP55,940 (1RS,3RS,4RS)-3-[hydroxy-2-(1,1-dimethylheptyl)phenyl]-4-(3-hydroxypropypcyclohexan-1-ol) at a dose of 1.25 mg/kg, in mice, according to the method described by R. G. Pertwee in Marijuana 84, Harvey D. J. eds, Oxford IRL Press, 263-277 (1985).

For example, compounds 5 and 7 showed a percentage inhibition of 28% and 32%, respectively, at 3 mg/kg po.

The antagonistic activity of the compounds was also shown by means of the model of inhibition of gastrointestinal transit induced by racemic CP55,940 (1RS,3RS,4RS-3-[hydroxy-2-(1,1-dimethylheptyl)phenyl]-4-(3-hydroxypropyl)cyclohexan-1-ol) in mice, according to the method described by Rinaldi-Carmona et al., J. Pharmacol, Exp. Ther. 2004, 310, 905-914. Briefly, male CD1 mice receive the test product per os 30 minutes or 2 hours before administration of the racemic CP55,940 agonist (1RS,3RS,4RS-3-[hydroxy-2-(1,1-dimethylheptyl)phenyl]-4-(3-hydroxypropyl)cyclohexan-1-ol) (0.15 mg/kg ip in 10% Cremophor). After a further 30 minutes, the animals receive a charcoal bolus po. Thirty minutes later, the animals are euthanized (CO₂/O₂) and the intestine is dissected. The progression of the charcoal bolus in the intestine is expressed as percentage of the total length of the intestine.

For example, compounds 5, 7, 34, 39 and 47 showed percentages of inhibition at 1 mg/kg of 82%, 68%, 46%, 66% and 68%, respectively, at 3 hours after administration of the product. Consequently, the compounds of the invention of formula (I) are cannabinoid CB1-type receptor antagonists in vitro and in vivo. Some compounds are active in vivo on both the hypothermia test and the transit test, and some compounds show dissociated activities between the hypothermia test and the transit test.

Hence the compounds according to the invention can be used in the treatment or prevention of diseases involving cannabinoid CB1 receptors. These compounds exhibit a peripheral activity dissociated from the central activity.

For example, and in a non-limiting manner, the compounds of formula (I) are of use as psychotropic medicaments, in particular for the treatment of psychiatric disorders including anxiety, depression, mood disorders, insomnia, delirium disorders, obsessive disorders, psychoses in general, schizophrenia, attention deficit hyperactivity disorders (ADHD) in hyperkinetic children (MBD), and also for the treatment of disorders associated with the use of psychotropic substances, in particular in the case of a substance abuse and/or a substance dependency, including alcohol dependency and nicotine dependency and withdrawal disorders. The compounds of formula (I) according to the invention can be used as medicaments for the treatment of migraine, stress, diseases of psychosomatic origin, panic attacks, epilepsy, motor disorders, in particular dyskinesia or Parkinson's disease, shaking and dystonia.

The compounds of formula (I) according to the invention can be used as medicaments for skin cancer and skin protection.

The compounds of formula (I) according to the invention can also be used as medicaments in the treatment of memory disorders, cognitive disorders, in particular in the treatment of cognitive disorders associated with senile dementia, with Alzheimer's disease, with schizophrenia and with neurodegenerative diseases, and also in the treatment of attention or alertness disorders.

Furthermore, the compounds of formula (I) may be of use as neuroprotectors, in the treatment of ischaemia and cranial traumas and the treatment of neurodegenerative diseases—including Huntington's chorea and Tourette's syndrome.

The compounds of formula (I) according to the invention can be used as medicaments in the treatment of pain: neuropathic pain, acute peripheral pain, chronic pain and pain of inflammatory origin.

The compounds of formula (I) according to the invention can be used as medicaments in the treatment of appetite disorders, appetence disorders (craving for sugars, carbohydrates, drugs, alcohols or any appetising substance) and/or eating disorders, in particular for the treatment of bulimia and also for the treatment of type II diabetes or non-insulin-dependent diabetes and for the treatment of dyslipidaemia and metabolic syndrome. Thus the compounds of formula (I) according to the invention are of use in the treatment of obesity and the risks associated with obesity, in particular cardiovascular risks.

Furthermore, the compounds of formula (I) according to the invention can be used as medicaments in the treatment of gastrointestinal disorders, diarrhoea disorders, ulcers, vomiting, bladder and urinary disorders, disorders of endocrine origin, cardiovascular disorders, hypotension, haemorrhagic shock, septic shock, cirrhosis, hepatic fibrosis, steatohepatitis and hepatic steatosis, irrespective of the etiology of these conditions: in particular, virus, alcohol, medicament, chemical product, autoimmune disease, obesity, diabetes, congenital metabolic disease (haemochromatosis, alpha-1 antitrypsin deficiency, Wilson's disease, etc.), chronic liver cirrhosis, fibrosis, non-alcoholic steatohepatitis (NASH), asthma, chronic obstructive pulmonary diseases, Raynaud's syndrome, glaucoma, fertility disorders, inflammatory phenomena, inflammatory diseases, immune system diseases, in particular autoimmune or neuroinflammatory diseases such as rheumatoid arthritis, reactive arthritis, diseases which result in demyelination, multiple sclerosis, infectious and viral diseases such as encephalitis, strokes, and also as medicaments for anti-cancer chemotherapy, for the treatment of Guillain-Barré syndrome and for the treatment of osteoporosis and sleep apnoea.

According to one of its aspects, the present invention relates to the use of a compound of formula (I), of the pharmaceutically acceptable salts thereof, and of the solvates or hydrates thereof, for the treatment of the disorders and diseases indicated above.

According to another of its aspects, the present invention relates to pharmaceutical compositions comprising, as active principle, a compound according to the invention. These pharmaceutical compositions contain an effective dose of at least one compound according to the invention, or a pharmaceutically acceptable salt of said compound, and also at least one pharmaceutically acceptable excipient.

Said excipients are selected, according to the pharmaceutical form and the method of administration desired, from the usual excipients which are known to those skilled in the art.

In the pharmaceutical compositions of the present invention for oral, sublingual, subcutaneous, intramuscular, intravenous, topical, local, intratracheal, intranasal, transdermal or rectal administration, the active principle of formula (I) above, or the salt thereof, can be administered in unit administration form, as a mixture with conventional pharmaceutical excipients for the treatment of the disorders or diseases mentioned above.

Suitable unit administration forms comprise oral forms such as tablets, soft or hard gel capsules, powders, granules and oral solutions or suspensions, sublingual, buccal, intratracheal, intraocular and intranasal administration forms, forms for administration by inhalation, topical, transdermal, subcutaneous, intramuscular or intravenous administration forms, rectal administration forms, and implants. For topical application, the compounds according to invention may be used in creams, gels, ointments or lotions.

By way of example, a unit administration form of a compound according to the invention in tablet form may comprise the following components:

| | |
|---|---|
| Compound according to the invention | 50.0 mg |
| Mannitol | 223.75 mg |
| Sodium croscarmellose | 6.0 mg |
| Maize starch | 15.0 mg |
| Hydroxypropylmethylcellulose | 2.25 mg |
| Magnesium stearate | 3.0 mg |

There may be specific cases where higher or lower dosages are appropriate; such dosages do not depart from the scope of the invention. According to customary practice, the dosage suitable for each patient is determined by the doctor according to the method of administration and the weight and response of said patient.

According to another of its aspects, the present invention also relates to a method of treating the pathologies indicated above, which comprises the administration, to a patient, of an effective dose of a compound according to the invention or of a pharmaceutically acceptable salt thereof.

1. A compound of formula (I) 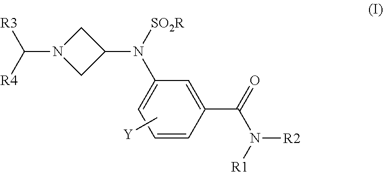
in which:
R represents a (C₁-C₆)alkyl group or a halo(C₁-C₆)alkyl group;
R1 represents a hydrogen atom;
R2 represents a
heteroaromatic group or a heteroaromatic (C₁-C₄)alkyl group, these groups being optionally substituted by one or more atoms or groups selected from a halogen, a hydroxyl, a cyano, oxo, NH₂, C(O)NH₂, a (C₁-C₆)alkyl group, a halo(C₁-C₆)alkyl group, a (C₁-C₆)alkoxy group, a halo(C₁-C₆)alkoxy group or a COO(C₁-C₆)alkyl group;
R3 and R4 represent independently of one another a phenyl group which is optionally substituted by one or more atoms or groups selected from a halogen, a cyano, a (C₁-C₆)alkyl group, a halo(C₁-C₆)alkyl group, a (C₁-C₆)alkoxy group or a halo(C₁-C₆)alkoxy group;
Y represents a hydrogen atom, a halogen, a cyano, a(C₁-C₆)alkyl group, a halo(C₁-C₆)alkyl group, a (C₁-C₆)alkoxy group, a halo(C₁-C₆)alkoxy group or a (C₁-C₆)alkylS(O)ₚ group;
p is between 0 and 2;
in the form of the base or an addition salt with an acid.

2. The compound according to claim 1,
wherein
R represents a methyl,
R3 and R4 each represent a phenyl group which is optionally substituted by a chlorine atom in para position,
Y represents a hydrogen atom or a halogen,
R1 represents a hydrogen atom,
R2 represents a
heteroaromatic group or a heteroaromatic (C₁-C₄)alkyl group, the heteroaromatic group representing a thiazole, imidazole, thiadiazole, pyridine, isoxazole, pyrimidine, pyrazole, oxadiazole, triazole or isothiazole which is optionally substituted by one or more of (C₁-C₆)alkyl, halogen, hydroxyl, amino, C(O)NH₂, halo(C₁-C₆)alkyl;
in the form of the base or an addition salt with an acid,

3. The compound according to claim 1, selected from:
3-[{1-[bis(4-chlorophenyl)methyl]azetidin-3-yl}(methylsulphonyl)amino]-N-(1,3-thiazol-2-yl)benzamide
3-[{1-[bis(4-chlorophenyl)methyl]azetidin-3-yl}(methylsulphonyl)amino]-N-[2-(1H-imidazol-1-yl)ethyl]benzamide
3-[{1-[bis(4-chlorophenyl)methyl]azetidin-3-yl}(methylsulphonyl)amino]-N-(1,3,4-thiadiazol-2-yl)benzamide
3-[{1-[bis(4-chlorophenyl)methyl]azetidin-3-yl}(methylsulphonyl)amino]-N-(4-hydroxy-1-methyl-1H-imidazol-2-yl)benzamide
3-[{1-[bis(4-chlorophenyl)methyl]azetidin-3-yl}(methylsulphonyl)amino]-N-(isoxazol-3-yl)benzamide
3-[{1-[bis(4-chlorophenyl)methyl]azetidin-3-yl}(methylsulphonyl)amino]-N-(5-cyclopropyl-1,3,4-thiadiazol-2-yl)benzamide
3-[{1-[bis(4-chlorophenyl)methyl]azetidin-3-yl}(methylsulphonyl)amino]-N-(pyridin-2-yl)benzamide
3-[{1-[bis(4-chlorophenyl)methyl]azetidin-3-yl}(methylsulphonyl)amino]-N-(pyrimidin-2-yl)benzamide
3-[{1-[bis(4-chlorophenyl)methyl]azetidin-3-yl}(methylsulphonyl)amino]-N-(1H-pyrazol-3-yl)benzamide
N-(4-amino-1,2,5-oxadiazol-3-yl)-3-[{1-[bis(4-chlorophenyl)methyl]azetidin-3-yl}(methylsulphonyl)amino]benzamide
3-[{1-[bis(4-chlorophenyl)methyl]azetidin-3-yl}(methylsulphonyl)amino]-N-(4-hydroxypyridin-2-yl)benzamide
3-[({3-[{1-[bis(4-chlorophenyl)methyl]azetidin-3-yl}(methylsulphonyl)amino]phenyl}-carbonyl)amino]-1H-pyrazole-4-carboxamide
3-[{1-[bis(4-chlorophenyl)methyl]azetidin-3-yl}(methylsulphonyl)amino]-N-(1H-1,2,4-triazol-3-ylmethyl)benzamide
3-[{1-[bis(4-chlorophenyl)methyl]azetidin-3-yl}(methylsulphonyl)amino]-N-(1H-pyrazol-3-ylmethyl)benzamide
3-[{1-[bis(4-chlorophenyl)methyl]azetidin-3-yl}(methylsulphonyl)amino]-N-[2-(1H-pyrazol-1-yl)ethyl]benzamide
3-[{1-[bis(4-chlorophenyl)methyl]azetidin-3-yl}(methylsulphonyl)amino]-N-[2-(pyrimidin-2-yl)ethyl]benzamide
3-[{1-[bis(4-chlorophenyl)methyl]azetidin-3-yl}(methylsulphonyl)amino]-5-fluoro-N-isoxazol-3-yl)benzamide
3-[{1-[bis(4-chlorophenyl)methyl]azetidin-3-yl}(methylsulphonyl)amino]-5-fluoro-N-(pyridin-2-yl)benzamide
3-[{1-[bis(4-chlorophenyl)methyl]azetidin-3-yl}(methylsulphonyl)amino]-5-fluoro-N-(1,3-thiazol-2-yl)benzamide
3-[{1-[bis(4-chlorophenyl)methyl]azetidin-3-yl}(methylsulphonyl)amino]-5-fluoro-N-(pyrimidin-2-yl)benzamide
3-[{1-[bis(4-chlorophenyl)methyl]azetidin-3-yl}(methylsulphonyl)amino]-5-fluoro-N-(5-methylisoxazol-3-yl)benzamide
3-[{1-[bis(4-chlorophenyl)methyl]azetidin-3-yl}(methylsulphonyl)amino]-5-fluoro-N-[4-(trifluoromethyl)-1,3-thiazol-2-yl]benzamide
3-[{1-[bis(4-chlorophenyl)methyl]azetidin-3-yl}(methylsulphonyl)amino]-5-fluoro-N-(3-methylisothiazol-5-yl)benzamide
3-[{1-[bis(4-chlorophenyl)methyl]azetidin-3-yl}(methylsulphonyl)amino]-5-fluoro-N-(isoxazol-4-yl)benzamide
3-[{1-[bis(4-chlorophenyl)methyl]azetidin-3-yl}(methylsulphonyl)amino]-5-fluoro-N-(4-methyl-1,3-thiazol-2-yl)benzamide
3-[{1-[bis(4-chlorophenyl)methyl]azetidin-3-yl}(methylsulphonyl)amino]-5-fluoro-N-(3-methylisoxazol-5-yl)benzamide
3-[{1-[bis(4-chlorophenyl)methyl]azetidin-3-yl}(methylsulphonyl)amino]-5-fluoro-N-(4-methylpyridin-2-yl)benzamide
3-[{1-[bis(4-chlorophenyl)methyl]azetidin-3-yl}(methylsulphonyl)amino]-5-fluoro-N-(6-methylpyridin-2-yl)benzamide
3-[{1-[bis(4-chlorophenyl)methyl]azetidin-3-yl}(methylsulphonyl)amino]-5-fluoro-N-(1H-pyrazol-3-yl)benzamide
N-(6-aminopyridin-3-yl)-3-[{1-[bis(4-chlorophenyl)methyl]azetidin-3-yl}(methylsulphonyl)amino]benzamide
N-(3-amino-1H-1,2,4-triazol-5-yl)-3-[{1-[bis(4-chlorophenyl)methyl]azetidin-3-yl}(methylsulphonyl)amino]benzamide
3-[{1-[bis(4-chlorophenyl)methyl]azetidin-3-yl}(methylsulphonyl)amino]-N-[(3-hydroxyisoxazol-5-yl)methyl]benzamide
3-[{1-[bis(4-chlorophenyl)methyl]azetidin-3-yl}(methylsulphonyl)amino]-N-(2H-tetrazol-5-ylmethyl)benzamide
3-({1-[bis(4-chlorophenyl)methyl]azetidin-3-yl}methylsulphonylamino)-N-(4-cyanopyridin-2-yl)-5-fluorobenzamide
3-({1-[bis(4-chlorophenyl)methyl]azetidin-3-yl}methylsulphonylamino)-5-fluoro-N-pyridin-2-ylmethylbenzamide
3-({1-[bis(4-chlorophenyl)methyl]azetidin-3-yl}methylsulphonylamino)-5-fluoro-N-(pyridin-3-ylmethyl)benzamide
3-({1-[bis(4-chlorophenyl)methyl]azetidin-3-yl}methylsulphonylamino)-5-fluoro-N-[1-(pyridin-3-yl)ethyl]benzamide
3-({1-[bis(4-chlorophenyl)methyl]azetidin-3-yl}methylsulphonylamino)-5-fluoro-N-[2-(pyridin-3-yl)propyl]benzamide
3-({1-[bis(4-chlorophenyl)methyl]azetidin-3-yl}methylsulphonylamino)-5-fluoro-N-[1-(pyridin-2-yl)ethyl]benzamide
3-({1-[bis(4-chlorophenyl)methyl]azetidin-3-yl}methylsulphonylamino)-5-fluoro-N-[(2-methyl-thiazol-4-yl)methyl]benzamide
3-({1-[bis(4-chlorophenyl)methyl]azetidin-3-yl}methylsulphonylamino)-5-fluoro-N-[2-([1,2,4]triazol-1-yl)propyl]benzamide
3-({1-[bis(4-chlorophenyl)methyl]azetidin-3-yl}methylsulphonylamino)-5-fluoro-N-[1-(2-methyl-thiazol-4-yl)ethyl]benzamide
3-({1-[bis(4-chlorophenyl)methyl]azetidin-3-yl}methylsulphonylamino)-5-fluoro-N-[1-(1-methyl-1H-pyrazol-4-yl)-ethyl]benzamide
3-({1-[bis(4-chlorophenyl)methyl]azetidin-3-yl}methylsulphonylamino)-5-fluoro-N-(pyridin-3-yl)benzamide
3-({1-[bis(4-chlorophenyl)methyl]azetidin-3-yl}methylsulphonylamino)-5-fluoro-N-[1-(2-pyrazol-1-yl)propyl]benzamide
3-({1-[bis(4-chlorophenyl)methyl]azetidin-3-yl}methylsulphonylamino)-5-fluoro-N-(pyridin-4-ylmethyl)benzamide
3-({1-[bis(4-chlorophenyl)methyl]azetidin-3-yl}methylsulphonylamino)-5-fluoro-N-[(6-oxo-1,6-dihydropyridin-3-yl)methyl]benzamide
3-({1-[bis(4-chlorophenyl)methyl]azetidin-3-yl}methylsulphonylamino)-5-fluoro-N-[(1-pyridin-4-yl)-ethyl]benzamide
3-({1-[bis(4-chlorophenyl)methyl]azetidin-3-yl}methylsulphonylamino)-N-[(6-dimethylaminopyridin-3-yl)methyl]-5-fluorobenzamide
3-({1-[bis(4-chlorophenyl)methyl]azetidin-3-yl}methylsulphonylamino)-5-fluoro-N-[2-(pyrazin-2-yl)propyl]benzamide
3-({1-[bis(4-chlorophenyl)methyl]azetidin-3-yl}methylsulphonylamino)-5-fluoro-N-(pyridin-4-yl)benzamide
3-({1-[bis(4-chlorophenyl)methyl]azetidin-3-yl}methylsulphonylamino)-5-fluoro-N-[2-hydroxy-2-(pyridin-4-yl)ethyl]benzamide.

4. (canceled)

5. A pharmaceutical composition comprising the compound of claim 1.

6. A method of treating or preventing psychiatric disorders, dependence on and withdrawal from a substance, tobacco withdrawal, cognitive disorders and attention disorders, and acute and chronic neurodegenerative diseases in a patient in need thereof comprising administering to said patient a therapeutically effective amount of the pharmaceutical composition of claim 5.

7. A method of treating or preventing metabolic disorders, appetence disorders, appetite disorders, obesity, diabetes, metabolic syndrome, dyslipidaemia or sleep apnoea in a patient in need thereof comprising administering to said patient a therapeutically effective amount of the pharmaceutical composition of claim 5.

8. A method of treating or preventing pain, neuropathic pain, or neuropathic pain induced by anti-cancer agents in a patient in need thereof comprising administering to said patient a therapeutically effective amount of the pharmaceutical composition of claim 5.

9. A method of treating or preventing gastrointestinal disorders, vomiting, ulcers, diarrhoea disorders, bladder and urinary disorders, disorders of endocrine origin, cardiovascular disorders, hypotension, haemorrhagic shock, septic shock, liver diseases, chronic liver cirrhosis, fibrosis, non-alcoholic steatohepatitis (NASH), steatohepatitis and hepatic steatosis, irrespective of the etiology of these conditions (alcohol, medicament, chemical product, autoimmune disease, obesity, diabetes, congenital metabolic disease) in a patient in need thereof comprising administering to said patient a therapeutically effective amount of the pharmaceutical composition of claim 5.

10. A method of treating or preventing diseases of the immune system, rheumatoid arthritis, demyelination, multiple sclerosis or inflammatory diseases in a patient in need thereof comprising administering to said patient a therapeutically effective amount of the pharmaceutical composition of claim 5.

11. A method of treating or preventing Alzheimer's disease, Parkinson's disease, schizophrenia or cognitive disorders associated with schizophrenia, with diabetes, with obesity or with metabolic syndrome in a patient in need thereof comprising administering to said patient a therapeutically effective amount of the pharmaceutical composition of claim 5.

12. A method of treating or preventing asthma, chronic obstructive pulmonary diseases, Raynaud syndrome, glaucoma or fertility disorders in a patient in need thereof comprising administering to said patient a therapeutically effective amount of the pharmaceutical composition of claim 5.

13. A method of treating or preventing infectious and viral diseases such as encephalitis, cerebral strokes, Guillain-Barré syndrome, osteoporosis or sleep apnoea and for anti-cancer chemotherapy in a patient in need thereof comprising administering to said patient a therapeutically effective amount of the pharmaceutical composition of claim 5.

14. A process for preparing the compound according to claim 1 for which R, R1, R2, R3, R4 and Y are as defined in claim 1, characterized in that: 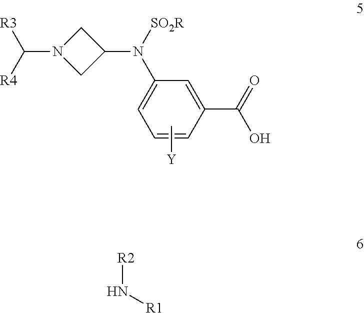
an acid derivative 5 and an amine derivative 6 are reacted in an inert solvent in the presence of a coupling agent and optionally of an additive preventing racemization, the product is deprotected if appropriate, and then the product is isolated and is optionally converted to an addition salt with an acid.
